SEQUENCE LISTING (15 sequences)

SEQ ID NO: 1 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 2 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLAFYQHKSMNEPKHGLALWEAKMAEKAREKEEECEKYG

SEQ ID NO: 3 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLCFYQHKSMNEPKHGLALWEAKMAEKAREKEEECEKYG

SEQ ID NO: 4 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLGFYQHKSMNEPKHGLALWEAKMAEKAREKEEECEKYG

SEQ ID NO: 5 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLIFYQHKSMNEPKHGLALWEAKMAEKAREKEEECEKYG

SEQ ID NO: 6 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLPFYQHKSMNEPKHGLALWEAKMAEKAREKEEECEKYG

SEQ ID NO: 7 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGKSIPVKILEAIFNTLEFVNNEELE

SEQ ID NO: 8 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGRSIPVKILEAIFNTLEFVNNEELE

SEQ ID NO: 9 - protein, 871 aa, Mus musculus
QSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 10 - protein, 507 aa, synthetic construct
GGSQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 11 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 12 - protein, 878 aa, synthetic construct
MGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 13 - protein, 9 aa, Homo sapiens
FSGVTACLD

SEQ ID NO: 14 - protein, 9 aa, synthetic construct
FSGVSACLD

SEQ ID NO: 15 - protein, 391 aa, synthetic construct
MNPQIRNPMERMYRDTFYDNFENEPILYGRSYTWLCYEVKIKRGRSNLLWDTGVFRGQVYFEPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQQFMPWYKFDENYAFLHRTLKEILRYLMEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

(19) United States
(12) Patent Application Publication
(10) Pub. No.: US 2026/0209842 A1
(43) Pub. Date: Jul. 23, 2026

(54) METHODS FOR ANALYSIS OF DNA METHYLATION

(71) Applicant: GUARDANT HEALTH, INC., Palo Alto, CA (US)

(72) Inventors: Andrew KENNEDY, San Diego, CA (US); James GHADIALI, San Diego, CA (US)

(21) Appl. No.: 19/371,623

(22) Filed: Oct. 28, 2025

Related U.S. Application Data
(63) Continuation of application No. PCT/US2024/027863, filed on May 3, 2024.
(60) Provisional application No. 63/499,934, filed on May 3, 2023.
(60) Provisional application No. 63/578,915, filed on Aug. 25, 2023.

(51) Int. Cl. C12Q 1/6869; C12Q 1/26; C12Q 1/34; C12Q 1/48; C12Q 1/6806; C12Q 1/6855; C12Q 1/6886

(52) U.S. Cl. CPC C12Q 1/6869; C12Q 1/26; C12Q 1/34; C12Q 1/48; C12Q 1/6806; C12Q 1/6855; C12Q 1/6886; C12Y 114/11; C12Y 204/01027; C12Y 305/04001; C12Q 2600/112; C12Q 2600/154; C12Q 2600/156

(57) ABSTRACT

Provided herein are methods of analyzing DNA molecules in a sample (e.g., including identifying methylated and hydroxymethylated cytosine positions).

### CROSS-REFERENCE TO RELATED APPLICATIONS

The present application is a Continuation of International Patent Application No. PCT/US2024/027863, filed May 3, 2024, which claims the benefit of priority of U.S. Provisional Patent Application No. 63/578,915, filed Aug. 25, 2023, and of U.S. Provisional Patent Application No. 63/499,934, filed May 3, 2023, which are incorporated by reference herein in their entireties for all purposes.

### SEQUENCE LISTING

The present application contains a sequence listing that has been submitted electronically in XML format. Said XML copy, created on Apr. 29, 2024, is named “GH0149WO.xml” and is 20,652 bytes in size. The information in the electronic format of the sequence listing is incorporated herein by reference in its entirety.

### INTRODUCTION AND SUMMARY

Single-nucleotide resolving assays to detect epigenetic variants or nucleoside modifications generally require a conversion of the modified nucleosides or corresponding unmodified nucleosides to change their base-pairing specificity. The conversion is then detected by sequencing. Examples of such methods include bisulfite and oxidative bisulfite and Tet-assisted bisulfite conversion, EM-seq, DM-seq, TAPS and TAPS β conversion, and ACE-seq. See, e.g., Moss et al., Nat Commun. 2018; 9:5068; WO2021/236778; Booth et al., Science 2012; 336:934-937; Yu et al., Cell 2012; 149:1368-80; Liu et al., Nature Biotechnology 2019; 37:424-429; Schutsky, E. K. et al.; and Vaisvila et al. Genome Research 2021 31 (7): 1280-1289.

EM-Seq methylation assays convert unmethylated cytosine to uracil, which is PCR-amplified and NGS-read as thymine. Incomplete (missed) conversion of an unmethylated base results in incorrectly identifying that base as methylated—i.e., a false positive signal. Conversely, erroneous conversion of methylated bases results in incorrectly identifying that base as unmethylated—i.e., a false negative signal. Given the risk of false positive and false negative signals in these assays, there is a need for improved methods that more accurately convert bases to reduce or avoid false positives and/or false negatives.

The presently disclosed methods can meet this need, provide other benefits, and/or at least provide the public with a useful choice. For example, provided herein is a method wherein DNA is contacted with one or more TET enzymes to oxidize 5-methylcytosine (5mC) present in the DNA. For at least a portion of the 5mC, the oxidation of 5mC results in formation of 5-formylcytosine (5-fC). The DNA is subsequently subjected to a further treatment that reduces the amount of 5-fC in the DNA and/or protects 5-hydroxymethylcytosine (5-hmC) that may be present in the DNA. In some embodiments, the treatment that reduces the amount of 5-fC in the DNA and/or protects 5-hydroxymethylcytosine (5-hmC) further oxidizes 5-fC in the DNA, e.g., to 5-caC (e.g., maximizes formation of 5-caC). In some embodiments, the treatment that reduces the amount of 5-fC in the DNA and/or protects 5-hmC comprises glucosylating or carbamoylating the 5-hmC. At least a portion of the DNA is then contacted with a cytosine deaminase, thereby converting unmethylated cytosine in the DNA to uracil, and producing treated DNA. At least a portion of the treated DNA is then sequenced. Without being bound by a particular theory, 5-caC is significantly less likely to be erroneously deaminated (e.g., by a cytosine deaminase, such as an APOBEC enzyme) than less oxidized modified cytosines (such as 5-fC and 5-hmC). Thus, reducing the amount of 5-fC in the DNA (such as by further oxidizing the 5-fC to 5-caC) and/or protecting 5-hmC in the DNA (such as by glucosylating or carbamoylating the 5-hmC), reduces the risk of false calls in which a base that was methylated (or hydroxymethylated) in the original DNA sample is identified as unmethylated because it was erroneously deaminated (e.g., even though it had been oxidized to 5-fC). Accordingly, methods are disclosed herein that can provide improved results by ensuring that the amount of 5-fC is low at the time of deamination, such as in comparison to the amount of 5-caC. For example, in some embodiments, the TET enzyme comprises a mutation (such as described elsewhere herein) that increases formation of 5-caC.

The disclosure includes the following exemplary embodiments.
Embodiment 1 is a method comprising:
a) contacting DNA with one or more TET enzymes to oxidize 5-methylcytosine (5mC) present in the DNA, wherein, for at least a portion of the 5mC, the oxidation of 5mC results in formation of 5-formylcytosine (5-fC) for at least a portion of the 5mC;
b) after a), subjecting the DNA to a further treatment that reduces the amount of 5fC in the DNA and/or protects 5-hydroxymethylcytosine that may be present in the DNA;
c) contacting at least a portion of the DNA with a cytosine deaminase, thereby converting unmethylated cytosine in the DNA to uracil, thereby producing treated DNA; and
d) sequencing at least a portion of the treated DNA.
Embodiment 2 is the method of embodiment 1, wherein the further treatment comprises contacting the DNA with β-glucosyltransferase (βGT).
Embodiment 3 is the method of embodiment 1, wherein the further treatment comprises contacting the DNA with β-glucosyltransferase (βGT) and then contacting the DNA with one or more TET enzymes to further oxidize the 5fC.
Embodiment 4 is the method of embodiment 1, wherein the further treatment comprises contacting the DNA with βGT and then contacting the DNA with an alkoxylamine, such as a C2-6 alkoxylamine, such as ethoxylamine (EtONH2).
Embodiment 5 is the method of embodiment 1, wherein the further treatment comprises contacting the DNA with a ruthenate such as KRuO4 and then contacting the DNA with an alkoxylamine, such as a C2-6 alkoxylamine, such as EtONH2.
Embodiment 6 is the method of embodiment 1, wherein the further treatment comprises contacting the DNA with a reducing agent such as NaBH4 and then contacting the DNA with βGT.
Embodiment 7 is the method of embodiment 1, wherein the further treatment comprises contacting the DNA with βGT and then contacting the DNA with Pinnick oxidation reagents.
Embodiment 8 is the method of embodiment 1, wherein the further treatment comprises contacting the DNA with a ruthenate such as KRuO4 and then contacting the DNA with Pinnick oxidation reagents.
Embodiment 9 is the method of embodiment 1, wherein the further treatment comprises contacting the DNA with 4-acetamido-2,2,6,6-tetramethylpiperidine-1-oxoammonium tetrafluoroborate (ACT+BF4−) and then contacting the DNA with Pinnick oxidation reagents.
Embodiment 10 is the method of the immediately preceding embodiment, wherein the Pinnick oxidation reagents comprise a chlorite salt and an alkene.
Embodiment 11 is the method of the immediately preceding embodiment, wherein the chlorite salt is sodium chlorite and/or the alkene is a branched C5-10 alkene, such as a branched C5-7 alkene, such as 2-methyl-2-butene.
Embodiment 12 is the method of embodiment 1, wherein the further treatment comprises contacting the DNA with 4-acetamido-2,2,6,6-tetramethylpiperidine-1-oxoammonium tetrafluoroborate (ACT+BF4−) and then contacting the DNA with an alkoxylamine, such as a C2-6 alkoxylamine, such as EtONH2.
Embodiment 13 is the method of embodiment 1, wherein the further treatment comprises contacting the DNA with one or more TET enzymes a second time after step a), wherein the method further comprises purifying the DNA between steps a) and b), optionally wherein the DNA is purified using solid phase reversible immobilization (SPRI).
Embodiment 14 is the method of any one of the preceding embodiments, wherein the one or more TET enzymes comprise TETv.
Embodiment 15 is the method of any one of the preceding embodiments, wherein the one or more TET enzymes comprise TETcd.
Embodiment 16 is the method of any one of the preceding embodiments, wherein the one or more TET enzymes comprise TET1.
Embodiment 17 is the method of any one of the preceding embodiments, wherein the one or more TET enzymes comprise TET2.
Embodiment 18 is the method of the immediately preceding embodiment, wherein the TET2 is T1372S TET2.
Embodiment 19 is the method of any one of the preceding embodiments, wherein the one or more TET enzymes comprise TET1 and TET2.
Embodiment 20 is the method of any one of the preceding embodiments, wherein the DNA is contacted with the one or more TET enzymes in the absence of βGT.
Embodiment 21 is the method of the immediately preceding embodiment, wherein the DNA is contacted with βGT after it was contacted with the one or more TET enzymes.
Embodiment 22 is the method of any one of the preceding embodiments, wherein the further treatment comprises contacting the DNA with one or more TET enzymes a second time after step a) and then contacting the DNA with β-glucosyltransferase (βGT).
Embodiment 23 is the method of any one of the preceding embodiments, wherein the one or more TET enzymes comprise a V1900 mutant TET.
Embodiment 24 is the method of the immediately preceding embodiment, wherein the V1900 mutant TET is a human V1900 mutant TET.
Embodiment 25 is the method of any one of embodiments 23-24, wherein the V1900 mutant TET is a V1900 mutant TET2.
Embodiment 26 is the method of any one of embodiments 23-25, wherein the V1900 mutant TET is a V1900A mutant TET.
Embodiment 27 is the method of the immediately preceding embodiment, wherein the V1900A mutant TET has at least 80%, 85%, 90%, 95%, 96%, 97%, 98%, or 99% sequence identity to SEQ ID NO: 2.
Embodiment 28 is the method of the immediately preceding embodiment, wherein the V1900A mutant TET comprises the sequence of SEQ ID NO: 2.
Embodiment 29 is the method of any one of embodiments 23-25, wherein the V1900 mutant TET is a V1900C mutant TET.
Embodiment 30 is the method of the immediately preceding embodiment, wherein the V1900C mutant TET has at least 80%, 85%, 90%, 95%, 96%, 97%, 98%, or 99% sequence identity to SEQ ID NO: 3.
Embodiment 31 is the method of the immediately preceding embodiment, wherein the V1900C mutant TET comprises the sequence of SEQ ID NO: 3.
Embodiment 32 is the method of any one of embodiments 23-25, wherein the V1900 mutant TET is a V1900G mutant TET.
Embodiment 33 is the method of the immediately preceding embodiment, wherein the V1900G mutant TET has at least 80%, 85%, 90%, 95%, 96%, 97%, 98%, or 99% sequence identity to SEQ ID NO: 4.
Embodiment 34 is the method of the immediately preceding embodiment, wherein the V1900G mutant TET comprises the sequence of SEQ ID NO: 4.
Embodiment 35 is the method of any one of embodiments 23-25, wherein the V1900 mutant TET is a V1900I mutant TET.
Embodiment 36 is the method of the immediately preceding embodiment, wherein the V1900I mutant TET has at least 80%, 85%, 90%, 95%, 96%, 97%, 98%, or 99% sequence identity to SEQ ID NO: 5.
Embodiment 37 is the method of the immediately preceding embodiment, wherein the V1900I mutant TET comprises the sequence of SEQ ID NO: 5.
Embodiment 38 is the method of any one of embodiments 23-25, wherein the V1900 mutant TET is a V1900P mutant TET.
Embodiment 39 is the method of the immediately preceding embodiment, wherein the V1900P mutant TET has at least 80%, 85%, 90%, 95%, 96%, 97%, 98%, or 99% sequence identity to SEQ ID NO: 6.
Embodiment 40 is the method of the immediately preceding embodiment, wherein the V1900P mutant TET comprises the sequence of SEQ ID NO: 6.
Embodiment 41 is a method comprising:
a) contacting DNA with one or more TET enzymes to oxidize 5-methylcytosine (5mC) present in the DNA, wherein, for at least a portion of the 5mC, the oxidation of 5mC results in formation of 5-carboxylcytosine (5-caC), wherein the one or more TET enzymes comprises a V1900A mutant TET or a V1900P mutant TET;
b) after a), contacting at least a portion of the DNA with a cytosine deaminase, thereby converting unmethylated cytosine in the DNA to uracil, thereby producing treated DNA; and
c) sequencing at least a portion of the treated DNA.
Embodiment 42 is the method of embodiment 41, wherein the V1900A mutant TET or V1900P mutant TET is a human V1900A mutant TET or a human V1900P mutant TET.
Embodiment 43 is the method of any one of embodiments 41-42, wherein the V1900A mutant TET or V1900P mutant TET is a V1900A mutant TET2 or a V1900P mutant TET2.
Embodiment 44 is the method of embodiment 41-43, wherein the one or more TET enzymes comprises a V1900A mutant TET.
Embodiment 45 is the method of the immediately preceding embodiment, wherein the V1900A mutant TET has at least 80%, 85%, 90%, 95%, 96%, 97%, 98%, or 99% sequence identity to SEQ ID NO: 2.
Embodiment 46 is the method of the immediately preceding embodiment, wherein the V1900A mutant TET comprises the sequence of SEQ ID NO: 2.
Embodiment 47 is the method of any one of embodiments 41-43, wherein the one or more TET enzymes comprises a V1900P mutant TET.
Embodiment 48 is the method of the immediately preceding embodiment, wherein the V1900P mutant TET has at least 80%, 85%, 90%, 95%, 96%, 97%, 98%, or 99% sequence identity to SEQ ID NO: 6.
Embodiment 49 is the method of the immediately preceding embodiment, wherein the V1900P mutant TET comprises the sequence of SEQ ID NO: 6.
Embodiment 50 is the method of any one of embodiments 41-49, wherein the DNA is not contacted with a glucosyltransferase concurrently with or after being contacted with the one or more TET enzymes.
Embodiment 51 is the method of any one of embodiments 41-49, wherein the DNA is not contacted with a glucosyltransferase.
Embodiment 52 is the method of any one of embodiments 41-51, wherein the DNA is not contacted with an alkoxylamine, such as a C2-6 alkoxylamine, such as ethoxylamine (EtONH2) concurrently with or after being contacted with the one or more TET enzymes.
Embodiment 53 is the method of any one of embodiments 41-51, wherein the DNA is not contacted with an alkoxylamine, such as a C2-6 alkoxylamine, such as ethoxylamine (EtONH2).
Embodiment 54 is the method of any one of embodiments 41-53, wherein the DNA is not contacted with a ruthenate such as KRuO4 concurrently with or after being contacted with the one or more TET enzymes.
Embodiment 55 is the method of any one of embodiments 41-53, wherein the DNA is not contacted with a ruthenate such as KruO4.
Embodiment 56 is the method of any one of embodiments 41-55, wherein the DNA is not contacted with a reducing agent such as NaBH4 concurrently with or after being contacted with the one or more TET enzymes.
Embodiment 57 is the method of any one of embodiments 41-55, wherein the DNA is not contacted with a reducing agent such as NaBH4.
Embodiment 58 is the method of any one of embodiments 41-57, wherein the DNA is not contacted with Pinnick oxidation reagents concurrently with or after being contacted with the one or more TET enzymes.
Embodiment 59 is the method of any one of embodiments 41-57, wherein the DNA is not contacted with Pinnick oxidation reagents.
Embodiment 60 is the method of any one of embodiments 41-59, wherein the DNA is not contacted with 4-acetamido-2,2,6,6-tetramethylpiperidine-1-oxoammonium tetrafluoroborate (ACT+BF4−) concurrently with or after being contacted with the one or more TET enzymes.
Embodiment 61 is the method of any one of embodiments 41-59, wherein the DNA is not contacted with 4-acetamido-2,2,6,6-tetramethylpiperidine-1-oxoammonium tetrafluoroborate (ACT+BF4−).
Embodiment 62 is the method of any one of embodiments 41-61, wherein the DNA is not subjected to a further treatment that reduces the amount of 5fC in the DNA and/or protects 5-hydroxymethylcytosine that may be present in the DNA concurrently with or after being contacted with the one or more TET enzymes.
Embodiment 63 is the method of any one of embodiments 41-61, wherein the DNA is not subjected to a further treatment that reduces the amount of 5fC in the DNA and/or protects 5-hydroxymethylcytosine that may be present in the DNA.
Embodiment 64 is the method of any one of the preceding embodiments, wherein the DNA is contacted with the one or more TET enzymes in a buffer having a pH of about 6.5, 6.6, 6.7, or 6.8, or a pH of 6.65+x where x is 0.7, 0.6, 0.5, 0.4, 0.3, 0.2, or 0.15.
Embodiment 65 is the method of any one of the preceding embodiments, wherein the DNA is contacted with the one or more TET enzymes in a buffer having a pH ranging from 6 to 7.
Embodiment 66 is the method of any one of the preceding embodiments, wherein the DNA is contacted with the one or more TET enzymes in a buffer having a pH ranging from 6.4 to 6.9.
Embodiment 67 is the method of any one of the preceding embodiments, wherein the DNA is contacted with the one or more TET enzymes in a buffer having a pH ranging from 6.5 to 6.8.
Embodiment 68 is the method of any one of the preceding embodiments, wherein the cytosine deaminase is an AID/APOBEC family cytosine deaminase enzyme, such as an APOBEC enzyme, optionally wherein the APOBEC enzyme is APOBEC3A.
Embodiment 69 is the method of any one of the preceding embodiments, wherein the APOBEC enzyme is HYPER-A3B-1.
Embodiment 69.1 is the method of any one of embodiments 1-67, wherein the cytosine deaminase is MsddA.
Embodiment 69.2 is the method of any one of embodiments 1-67, wherein the cytosine deaminase is AshDa01.
Embodiment 70 is the method of any one of the preceding embodiments, wherein the DNA comprises a first member of a binding pair, optionally wherein the first member of the binding pair is biotin.
Embodiment 71 is the method of the immediately preceding embodiment, wherein the first member of the binding pair is attached to an adapter joined to the DNA.
Embodiment 72 is the method of embodiment 70 or 71, wherein the DNA is bound to a solid phase comprising a second member of the binding pair during steps a)-b), optionally wherein the solid phase comprises beads and/or the second member of the binding pair is streptavidin.
Embodiment 73 is the method of the immediately preceding embodiment, wherein the DNA is released from the solid phase before step c).
Embodiment 73.1 is the method of embodiment 73, wherein the method further comprises amplifying the DNA between step (b) and step (c), and prior to a step of releasing the DNA from the solid phase.
Embodiment 73.2 is the method of the immediately preceding embodiment, further comprising separating the amplified DNA from the DNA bound to the solid phase prior to step (c), thereby contacting the DNA bound to the solid phase, but not the amplified DNA, with a cytosine deaminase in step (c).
Embodiment 73.3 is the method of the immediately preceding embodiment, wherein the DNA bound to the solid phase is released from the solid phase prior to contacting the DNA with the cytosine deaminase.
Embodiment 73.4 is the method of any one of embodiment 73.1-73.3, further comprising comparing sequence data obtained in step (d) with sequence data obtained by sequencing the amplified DNA that was not contacted with the cytosine deaminase; and identifying point differences between the converted DNA sequences and the or non-converted DNA sequence data as nucleosides having a modification status that permits a change in base pairing specificity on exposure to the cytosine deaminase.
Embodiment 74 is the method of any one of embodiments 73-73.4, wherein the DNA is released from the solid phase by cleaving a cleavable moiety in an adapter joined to the DNA, optionally wherein the cleavable moiety comprises uracil or photocleavable biotin.
Embodiment 75 is the method of any one of the preceding embodiments, wherein the method further comprises enriching the DNA by capturing a target region set from the sample, wherein the capture step is before, after or in between step (a) and step (c).
Embodiment 76 is the method of any one of the preceding embodiments, further comprising
a) comparing the sequence data obtained in step (d) with
   (A) a pre-determined reference sequence; and/or
   (B) sequence data obtained by sequencing a sub-sample of the DNA that was not subjected to a conversion procedure; and
b) identifying point differences between the converted DNA sequences and the reference sequence (A) or non-converted DNA sequence data (B) as nucleosides having a modification status that permits a change in base pairing specificity on exposure to the cytosine deaminase.
Embodiment 76.1 is the method of any one of embodiments 1-75, further comprising
a) comparing the sequence data obtained in step (d) with
   (A) a pre-determined reference sequence; and/or
   (B) sequence data obtained by sequencing a sub-sample of the DNA that was not subjected to the conversion procedure; and
b) identifying point differences between the converted DNA sequences and the reference sequence (A) or non-converted DNA sequence data (B) as nucleosides having a modification status that permits a change in base pairing specificity on exposure to the cytosine deaminase.
Embodiment 77 is the method of any one of the preceding embodiments, wherein the DNA comprises cell-free DNA (cfDNA), optionally cfDNA obtained from a test subject, optionally wherein the test subject is a patient having or suspected of having cancer.
Embodiment 78 is the method of any one of the preceding embodiments, further comprising using the detection of modified nucleosides in the DNA sample to determine or predict the presence of DNA produced by a cancer cell or tumor, to determine the probability that a test subject has a tumor or cancer, or to characterize a cancer or tumor of the subject.
Embodiment 79 is the method of any one of the preceding embodiments, wherein a non-converted subsample of the DNA is not contacted with the cytosine deaminase before sequencing, wherein the treated subsample and the non-converted subsample have different adapter sequences, and wherein the converted subsample and the non-converted subsample are recombined before sequencing step (d).
Embodiment 80 is the method of any one of the preceding embodiments, further comprising analyzing the DNA to detect copy number variation, single nucleotide variants, insertions, deletions, methylation, and/or fusions.
Embodiment 81 is the method of any one of the preceding embodiments, further comprising analyzing the DNA to detect methylation and one or more of copy number variation, single nucleotide variants, insertions, deletions, and/or fusions.
Embodiment 82 is the method of any one of the preceding embodiments, further comprising capturing epigenetic target regions from the adapter-ligated DNA and amplifying and sequencing the epigenetic target regions.
Embodiment 83 is the method of embodiment 82, wherein the captured epigenetic target regions form an epigenetic target region set.
Embodiment 84 is the method of embodiment 83, wherein the epigenetic target region set comprises a plurality of type-specific epigenetic target regions, and wherein the type-specific epigenetic target regions are type-specific differentially methylated regions and/or type specific fragments.
Embodiment 85 is the method of embodiment 84, wherein the plurality of type-specific epigenetic target regions comprises type-specific hypomethylated regions.
Embodiment 86 is the method of any one of the preceding embodiments, wherein the sample is a blood sample.
Embodiment 87 is the method of any one of embodiments 84-86, wherein the plurality of type-specific epigenetic target regions comprises target regions that are:
hypermethylated in immune cells relative to non-immune cell types present in the blood sample;
differentially methylated in colon relative to other tissue types;
differentially methylated in breast relative to other tissue types;
differentially methylated in liver relative to other tissue types;
differentially methylated in kidney relative to other tissue types;
differentially methylated in pancreas relative to other tissue types;
differentially methylated in prostate relative to other tissue types;
differentially methylated in skin relative to other tissue types; or differentially methylated in bladder relative to other tissue types.
Embodiment 88 is the method of any one of embodiments 84-87, wherein the plurality of type-specific epigenetic target regions comprises:
target regions that are hypomethylated in non-immune blood cells relative to the methylation level of the target regions in a different cell or tissue type in the sample;
fragments specific to immune cells relative to non-immune cell types present in the blood sample; or
fragments specific to colon, lung, breast, liver, kidney, pancreas, prostate, skin, or bladder relative to other tissue types.
Embodiment 89 is the method of any one of embodiments 84-88, further comprising identifying at least one cell type or tissue type from which the type-specific epigenetic target regions originated.
Embodiment 90 is the method of any one of embodiments 84-89, wherein the level of type-specific epigenetic target regions that originated from a cell or tissue type is determined.
Embodiment 91 is the method of the immediately preceding embodiment, wherein the levels of type-specific epigenetic target regions that originated from immune cells, non-immune blood cells, colon, lung, breast, liver, kidney, prostate, skin, bladder, or pancreas are determined.
Embodiment 92 is the method of embodiment 90 or embodiment 91, wherein the type-specific epigenetic target regions comprise cell-type specific, tissue-type specific, and/or cancer-type specific epigenetic target regions.
Embodiment 93 is the method of any one of embodiments 86-92, wherein the blood sample is fractionated prior to capturing at least an epigenetic target region set of DNA.
Embodiment 94 is the method of any one of the preceding embodiments, further comprising detecting the presence or absence of sequence variations and/or determining fragmentation patterns, wherein adapted DNA comprising quality control nucleosides indicative of sub-optimal or erroneous conversion of quality control nucleosides is included in detecting the presence or absence of sequence variations and/or determining fragmentation patterns.
Embodiment 95 is the method of any one of the preceding embodiments, wherein oligonucleotide adapters are ligated to the DNA.
Embodiment 96 is the method of the immediately preceding embodiment, wherein the oligonucleotide adapters are ligated to the DNA before step a).
Embodiment 97 is the method of embodiment 95 or 96, wherein the oligonucleotide adapters comprise sequencing primer binding sites.
Embodiment 98 is the method of any one of the preceding embodiments, wherein the method further comprises amplifying the DNA using primers targeting the adapters, wherein the amplifying step is between step (c) and the sequencing step (d).
Embodiment 99 is the method of any one of the preceding embodiments, wherein one or more nucleosides of the adapter is a modified nucleoside, optionally wherein the modified nucleoside comprises a modified cytosine.
Embodiment 100 is the method of the immediately preceding embodiment, wherein the modified nucleoside is 5-carboxyl cytosine, pyrrolo cytosine, or 5-propynyl cytosine.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

FIG. 1 illustrates exemplary workflows according to certain embodiments of the disclosure involving contacting DNA with TET followed sequentially by contacting the DNA with β-glucosyltransferase (βGT) and/or contacting the DNA with TET a second time, followed by deamination (e.g., using an APOBEC such as APOBEC3A).

FIG. 2 illustrates exemplary workflows according to certain embodiments of the disclosure involving contacting DNA with TET followed sequentially by contacting the DNA with NaBH₄, β-glucosyltransferase (βGT), and/or EtONH₂, followed by deamination (e.g., using an APOBEC such as APOBEC3A).

FIG. 3 illustrates exemplary workflows according to certain embodiments of the disclosure involving contacting immobilized DNA with reagents such as TET.

FIG. 4 illustrates an exemplary workflow according to certain embodiments of the disclosure involving contacting immobilized DNA with reagents such as TET wherein an amplified library is produced prior to treatment with a deaminase (e.g., an APOBEC such as APOBEC3A) and used for calling of genetic variants such as point mutations, indels, etc., and then immobilized DNA is subjected to deamination (e.g., using an APOBEC such as APOBEC3A).

FIG. 5 illustrates an exemplary workflow according to certain embodiments of the disclosure involving contacting DNA with TET followed sequentially by contacting the DNA with β-glucosyltransferase (βGT) such as T4 βGT and then with EtONH₂, followed by deamination (e.g., using an APOBEC such as APOBEC3A).

FIG. 6 illustrates an exemplary workflow according to certain embodiments of the disclosure involving contacting DNA with TET followed sequentially by contacting the DNA with β-glucosyltransferase (βGT) such as T4 βGT and then with Pinnick oxidation reagents, followed by deamination (e.g., using an APOBEC such as APOBEC3A).

FIG. 7 illustrates an exemplary workflow according to certain embodiments of the disclosure involving contacting DNA with TET followed sequentially by contacting the DNA with NaBH₄ and then with β-glucosyltransferase (βGT) such as T4 βGT, followed by deamination (e.g., using an APOBEC such as APOBEC3A).

FIG. 8 illustrates exemplary workflows according to certain embodiments of the disclosure involving contacting DNA with reagents such as TET followed sequentially by reagents selected from KRuO₄, Pinnick oxidation reagents, EtONH₂, and ACT⁺BF₄⁻.

FIG. 9 is a schematic diagram of an example of a system suitable for use with some embodiments of the disclosure.

### DETAILED DESCRIPTION

Reference will now be made in detail to certain embodiments of the disclosure. While the disclosure will be described in conjunction with such embodiments, it will be understood that they are not intended to limit the invention to those embodiments. On the contrary, the disclosure is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids.

Numeric ranges are inclusive of the numbers defining the range. Measured and measurable values are understood to be approximate, taking into account significant digits and the error associated with the measurement.

Unless specifically noted in the above specification, embodiments in the specification that recite “comprising” various components are also contemplated as “consisting of” or “consisting essentially of” the recited components.

The section headings used herein are for organizational purposes and are not to be construed as limiting the disclosed subject matter in any way.

All patents, patent applications, websites, other publications or documents and the like cited herein whether supra or infra, are expressly incorporated by reference in their entirety for all purposes to the same extent as if each individual item were specifically and individually indicated to be so incorporated by reference. If different versions of a publication, website or the like are published at different times, the version most recently published at the effective filing date of the application is meant, unless otherwise indicated.

### Definitions

As used herein, “base pairing specificity” refers to the standard DNA base (A, C, G, or T) for which a given base most preferentially pairs. Thus, for example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G, for example, is irrelevant because uracil nonetheless most preferentially pairs with A among the four standard DNA bases.

Nucleosides of the “same identity” or “same nucleoside identity” refer to nucleosides with the same base, regardless of modification status of that base. For example, cytosine is considered to be the “same identity” as 5-methylcytosine (5mC) and/or 5-hydroxymethyl-cytosine (5hmC), despite them having different modification statuses.

A “conversion reagent” refers to a reagent that can be used to change the base pairing specificity of at least one modified or unmodified nucleoside in a nucleic acid. For example, APOBEC3A is a conversion reagent that can be used to change methylated cytosines (having base pairing specificity for G) to uracils (having base pairing specificity for A). A conversion procedure is a procedure that uses one or more conversion reagents to change the base pairing specificity of at least one modified or unmodified nucleoside in a nucleic acid.

A conversion reagent or procedure is “substantially not capable of changing the base pairing specificity” of a nucleoside with a first modification status (which may be modified or unmodified) if the conversion reagent or procedure preferentially changes the base pairing specificity of that nucleoside with a second, different modification status to the point that changes to the base pairing specificity of the nucleoside having the first modification status are properly considered erroneous. For example, DM-seq conversion is substantially not capable of changing the base pairing specificity of unmodified C (nucleoside with a first modification status) but preferentially changes the base pairing specificity of methylated cytosine (nucleoside with a second modification status).

“Capturing” one or more target nucleic acids refers to preferentially isolating or separating the one or more target nucleic acids from non-target nucleic acids.

A “captured set” of nucleic acids refers to nucleic acids that have undergone capture.

A “target-region set” or “set of target regions” refers to a plurality of genomic loci targeted for capture and/or targeted by a set of probes (e.g., through sequence complementarity).

“Corresponding to a target region set” means that a nucleic acid, such as cfDNA, originated from a locus in the target region set or specifically binds one or more probes for the target-region set.

“Sequence-variable target regions” refer to target regions that may exhibit changes in sequence such as nucleotide substitutions (i.e., single nucleotide variations), insertions, deletions, or gene fusions or transpositions in neoplastic cells (e.g., tumor cells and cancer cells) relative to normal cells. A sequence-variable target region set is a set of sequence-variable target regions. In some embodiments, the sequence-variable target regions are target regions that may exhibit changes that affect less than or equal to 50 contiguous nucleotides, e.g., less than or equal to 40, 30, 20, 10, 5, 4, 3, or 2 nucleotides, or that affect 1 nucleotide.

“Epigenetic target regions” refers to target regions that may show sequence-independent changes across tissue types (e.g., a target region having a different extent of methylation in a solid tissue type than in hematopoietic cells) or differences in neoplastic cells, such as tumor cells or cancer cells, relative to normal cells. In some embodiments, epigenetic target regions show sequence-independent differences in cfDNA originating from tissue types that ordinarily do not substantially contribute to cfDNA, such as lung, colon, etc., relative to background cfDNA, such as cfDNA that originated from hematopoietic cells. In some embodiments, epigenetic target regions show sequence-independent differences in cfDNA from subjects having cancer relative to cfDNA from healthy subjects. Examples of sequence-independent changes include, but are not limited to, changes in methylation (increases or decreases), nucleosome distribution, cfDNA fragmentation patterns, CCCTC-binding factor (“CTCF”) binding, transcription start sites, and regulatory protein binding regions. An epigenetic target region set is a set of epigenetic target regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasia-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed above than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions.

As used herein, an “epigenetic feature” refers to any feature of DNA or chromatin other than primary sequence (i.e., the sequence of A, C, G, and T bases). Epigenetic features include covalent modifications of bases, such as methylation, and modifications and positioning of histones and other stably DNA-associated proteins.

As used herein, a “differentially methylated region” refers to a region having a detectably different degree of methylation in at least one type of tissue relative to the degree of methylation in cell-free DNA from a healthy subject. In some embodiments, a differentially methylated region of DNA has a detectably different degree of methylation in at least one type of tissue relative to the degree of methylation in another type of tissue; or in a sample from a healthy subject relative to the degree of methylation in a subject having pre-cancer, cancer, or a neoplasm. In some embodiments, a differentially methylated region has a detectably higher degree of methylation in at least one type of tissue relative to the degree of methylation in cell-free DNA from a healthy subject. In some embodiments, a differentially methylated region has a detectably lower degree of methylation in at least one type of tissue relative to the degree of methylation in cell-free DNA from a healthy subject. In some embodiments, differentially methylated regions are hypomethylated in the erythrocyte lineage or in an immature red blood cell (e.g., reticulocyte) and hypermethylated in at least one non-erythrocyte cell or tissue type (e.g., a leukocyte or a solid tissue cell type, such as epithelial cells, muscle cells, etc.).

As used herein, “type-specific” in the context of an epigenetic variation means an epigenetic variation that is present at a detectably different degree in one cell or tissue type, or in a plurality of related cell or tissue types, relative to other cell or tissue types. Similarly, a “type-specific epigenetic target region” is an epigenetic target region that has a detectably different epigenetic characteristic in one cell or tissue type, or in a plurality of related cell or tissue types, relative to other cell or tissue types. Exemplary epigenetic characteristics are discussed in the definition of epigenetic target regions set forth above. For example, a “type-specific differentially methylated region” is a region of DNA that has a detectably different degree of methylation in one cell or tissue type, or in a plurality of related cell or tissue types, relative to other cell or tissue types. Examples of a type-specific differentially methylated region include tissue-specific differentially methylated regions, including those associated with copy-number gain in early cancer. In some embodiments, capturing, identification, and/or detection of type-specific differentially methylated regions facilitates identification of the cell or tissue type from which the DNA originated. The cell or tissue from which a type-specific differentially methylated region originated may be a wild type cell or tissue or a neoplastic cell or tissue. In another example, a “type-specific fragment” of DNA is a DNA fragment arising from a type-specific fragmentation pattern that is present at a detectably different degree in one cell or tissue type, or in a plurality of related cell or tissue types, relative to other cell or tissue types. In some embodiments, a type-specific fragment is only present in the specific cell or tissue type(s). In some embodiments, a type-specific fragment is present to a detectably greater extent in the specific cell or tissue type(s).

As used herein, a “blood sample” refers to a sample comprising whole blood or a component thereof (e.g., plasma, serum, buffy coat, plasma pellet).

“Buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

As used herein, “primer-annealed DNA” means DNA to which at least one primer is annealed.

As used herein, “partitioning” of nucleic acids, such as DNA molecules, means separating, fractionating, sorting, or enriching a sample or population of nucleic acids into a plurality of subsamples or subpopulations of nucleic acids based on one or more modifications or features that is in different proportions in each of the plurality of subsamples or subpopulations. Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, the form of the “originally isolated” sample refers to the composition or chemical structure of a sample at the time it was isolated and before undergoing any procedure that changes the chemical structure of the isolated sample. Similarly, a feature that is “originally present” in a molecule refers to a feature present in an “original molecule” or in molecules “originally comprising” the feature before the molecule undergoes any procedure that changes the chemical structure of the molecule.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

As used herein, a “label” is a capture moiety, fluorophore, oligonucleotide, or other moiety that facilitates detection, separation, or isolation of that to which it is attached.

As used herein, a “capture moiety” is a molecule that allows affinity separation of molecules linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “capture probe” means a probe comprising a capture moiety and that is generated by amplification and thus comprises an amplicon of the template DNA. In some embodiments, the amplification comprises polymerase chain reaction (PCR).

As used herein, “anti-parallel orientation” of two primers means that the primers anneal to a nucleic acid in opposite orientations relative to each other (e.g., to opposite strands of the nucleic acid) and/or in an orientation that is compatible with exponential PCR amplification. For example, primers that anneal to a rearrangement in an anti-parallel orientation can facilitate amplification of an amplicon comprising the rearrangement breakpoint.

As used herein, a “tag” is a molecule, such as a nucleic acid, label, fluorophore, or peptide, containing information that indicates a feature of the molecule to which the tag is associated. For example, molecules can bear a sample tag (which distinguishes molecules in one sample from those in a different sample), a molecular tag/molecular barcode/barcode (which distinguishes different molecules from one another (in both unique and non-unique tagging scenarios), a purification tag, and/or a detectable tag or label.

“Specifically binds” in the context of an probe or other oligonucleotide and a target sequence means that under appropriate hybridization conditions, the oligonucleotide or probe hybridizes to its target sequence, or replicates thereof, to form a stable probe: target hybrid, while at the same time formation of stable probe: non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein).

A nucleic acid is “produced by a tumor” or ctDNA or circulating tumor DNA, if it originated from a tumor cell. Tumor cells are neoplastic cells that originated from a tumor, regardless of whether they remain in the tumor or become separated from the tumor (as in the cases, e.g., of metastatic cancer cells and circulating tumor cells).

A “target region” in the context of a nucleic acid refers to a genomic locus targeted for identification and/or capture, for example, by using probes (e.g., through sequence complementarity). A “target region set” or “set of target regions” refers to a plurality of genomic loci targeted for identification and/or capture, for example, by using a set of probes (e.g., through sequence complementarity).

The “capture yield” of a collection of probes for a given target region set refers to the amount (e.g., amount relative to another target region set or an absolute amount) of nucleic acid corresponding to the target region set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

The term “methylation” or “DNA methylation” refers to addition of a methyl group to a nucleotide base in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence)). In some embodiments, DNA methylation refers to addition of a methyl group to adenine, such as in N⁶-methyladenine. In some embodiments, DNA methylation is 5-methylation (modification of the carbon in the 5th position of the cytosine ring). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the carbon in the 3^{rd} position of the cytosine ring). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

“Methyltransferases” or “MTases” are a large group of enzymes that methylate their substrates but can be split into several subclasses based on their structural features. The most common class of methyltransferases is class I, all of which contain a Rossmann fold for binding S-Adenosyl-L-methionine. Examples of methyltransferases include, but are not limited to, CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), and DNA adenine methyltransferase (Dam). An exemplary carboxymethyltransferase for use in the disclosed methods is Mycoplasma penetrans CpG carboxymethyltransferase (M.Mpel) of SEQ ID NO: 7 or SEQ ID NO: 8. SEQ ID NO: 7 has an N374K substitution relative to wild-type M.Mpel. SEQ ID NO: 8 has an N374R substitution relative to wild-type M.Mpel. See, e.g., WO2021236778A2. Polypeptides comprising sequences having at least 90, 92, 94, 96, 97, 98 or 99% sequence identity with SEQ ID NO: 7 or SEQ ID NO: 8, optionally wherein the amino acid residue corresponding to position 374 of SEQ ID NO: 7 is R or K, are also within the scope of the disclosure. Also included are homologous cytosine methyltransferases which can be genetically engineered to utilize CxSAM as a substrate. Such enzymes include for example Dcm or a GpC MTase such as M.CviPI.

The “modified nucleoside profile of DNA” means the position and identity of the nucleoside and the modification status of the nucleoside, such as methylations, within a DNA sequence. As described above, enzymatic conversion followed by sequencing detect one or more different types of modified or unmodified nucleoside. For example, the DM-seq method detects 5-methylcytosine (5mC). Hence, a method for analyzing the modified nucleoside profile of DNA in a sample typically means identifying particular modifications or groups of modification, such as 5mC. Modified nucleosides are identified according to the specific method/conversion procedure being used as described above. This generally involves comparing sequence data obtained from DNA that has been subjected to a conversion procedure to a reference sequence. Typically, the method involves (i) comparing the sequence data with (A) one or more pre-determined reference sequence, typically corresponding to one or more epigenetic target regions where particular significance is attached to the modified nucleoside profile, e.g. in diagnosing, prognosing or characterizing a cancer; or (B) sequence data obtained by sequencing a sub-sample of the DNA that was not subjected to the conversion procedure, for example a subsample that was separated before subjecting a separate subsample to the conversion procedure, for example as described herein; and (ii) identifying point differences between the converted DNA sequences and the reference sequence(s) (A) or non-converted DNA sequences (B) as nucleosides (in the initial sample) having a modification status that permits a change in base pairing specificity on exposure to the conversion procedure.

It is recognized that the modified nucleoside profile determined by standard conversion and sequencing methods may contain errors due to incomplete or erroneous conversion of modified or unmodified nucleosides in the sample. The method of the disclosure provides a means for assessing the conversion rate on either a sample or molecular basis.

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues. As used herein, “type-specific hypermethylation” means an increased level or degree of methylation of DNA in at least one cell or tissue type, or in a plurality of related cell or tissue types, relative to other cell or tissue types. In some embodiments, capturing, identification, and/or detection of type-specific hypermethylated regions facilitates identification of the cell or tissue type from which the DNA originated. The cell or tissue from which a type-specific hypermethylated region originated may be a wild type cell or tissue or a neoplastic cell or tissue.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues. As used herein, “type-specific hypomethylation” means a decreased level or degree of methylation of DNA in at least one cell or tissue type, or in a plurality of related cell or tissue types, relative to other cell or tissue types. In some embodiments, capturing, identification, and/or detection of type-specific hypomethylated regions facilitates identification of the cell or tissue type from which the DNA originated. The cell or tissue from which a type-specific hypomethylated region originated may be a wild type cell or tissue or a neoplastic cell or tissue.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine.

A “modified nucleoside” is a nucleoside that comprises a difference in chemical structure from an unmodified nucleoside. In the case of DNA, an unmodified nucleoside comprises a deoxyribosyl and one of adenine, cytosine, guanine, or thymine. In some embodiments, a modified nucleoside comprises a modified cytosine. In some embodiments, a modified nucleoside comprises a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded. In other embodiments, a modified nucleoside comprises 5-carboxyl cytosine, 5-carboxymethyl cytosine, pyrrolo cytosine, or a 5-(C₂₋₆ alkynyl) cytosine such as 5-propynyl cytosine. In some embodiments, a modified nucleoside of an adapter disclosed herein is a quality control nucleoside. In some embodiments, a modified nucleoside of an adapter disclosed herein is not a quality control nucleoside.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

A “X₁nnnX₂ mutation” in a specified polypeptide as used herein, where X₁ and X₂ are amino acids and nnn is a position in an amino acid sequence, refers to a substitution in the polypeptide of amino acid X₁ present at position nnn of the full-length wild-type polypeptide with amino acid X₂. The polypeptide is the human polypeptide unless indicated otherwise. The polypeptide comprising the X₁nnnX₂ mutation may, but does not necessarily, comprise additional differences from the wild-type sequence, including but not limited to truncations and deletions as well as other substitutions. For example, a “T1372S mutation” in TET2 refers to a substitution in a TET2 enzyme of the threonine present at position 1372 of the full-length wild-type human TET2 enzyme with a serine. Position 1372 of wild-type human TET2 aligns to position 258 and 248, respectively, of the truncated TET2 sequences disclosed as SEQ ID NOs: 23 and 24 of U.S. Pat. No. 10,961,525. The immediate wild-type sequence context of position 1372 of human TET2 is FSGVTACLD (SEQ ID NO: 13) where the Tis at position 1372. Thus, a TET2 enzyme comprising a T1372S mutation may comprise the sequence FSGVSACLD (SEQ ID NO: 14) or optionally a variant of SEQ ID NO: 14 in which at least 5, 6, 7, or 8 positions match SEQ ID NO: 14 including position 5. Similarly, a “V1900X₂ mutation” where X₂ is A, C, G, I, or P in TET2 refers to a substitution in a TET2 enzyme of the valine present at position 1900 of the full-length wild-type human TET2 enzyme with an alanine, cysteine, glycine, isoleucine, or proline.

“Or” is used in the inclusive sense, i.e., equivalent to “and/or,” unless the context requires otherwise.

### Exemplary Methods and Reagents

Provided herein is a method comprising (a) contacting DNA with one or more TET enzymes to oxidize 5-methylcytosine (5mC) present in the DNA, wherein, for at least a portion of the 5mC, the oxidation of 5mC results in formation of 5-formylcytosine (5-fC) for at least a portion of the 5mC; (b) subjecting the DNA to a further treatment that reduces the amount of 5fC in the DNA and/or protects 5-hydroxymethylcytosine that may be present in the DNA; (c) contacting at least a portion of the DNA with a cytosine deaminase, thereby converting unmethylated cytosine in the DNA to uracil, thereby producing treated DNA; and (d) sequencing at least a portion of the treated DNA. The method may be performed in order from (a)-(d). In some such embodiments, all or substantially all of the oxidized 5mCs (e.g., the 5fCs, and/or 5caCs) and/or the protected 5hmCs in the DNA are not converted by the cytosine deaminase. Thus, in some embodiments, cytosines that were methylated (and/or cytosines that were hydroxymethylated) in the original DNA sample are read as Cs in an analysis of the sequencing data, whereas cytosines that were unmethylated in the original DNA sample are read as Ts.

TET enzymes can oxidize 5mC to 5hmC, 5fC, and 5caC. In this process, 5mC is first oxidized to 5hmC, which subsequently undergoes iterative oxidation to 5fC and 5caC. Various TET enzymes may be used in the disclosed embodiments. In some embodiments, the one or more TET enzymes comprise TETv. TETv is described in U.S. Pat. No. 10,260,088 and its sequence is SEQ ID NO: 1 therein (SEQ ID NO: 10 in the present application). In some embodiments, the one or more TET enzymes comprise TETcd. TETcd is described in U.S. Pat. No. 10,260,088 and its sequence is SEQ ID NO: 3 therein (SEQ ID NO: 9 in the present application). In some embodiments, the one or more TET enzymes comprise TET1. In some embodiments, the one or more TET enzymes comprise TET2. In some embodiments, the one or more TET enzymes comprise TET1 and TET2. In some embodiments, the TET2 is T1372S TET2. In some embodiments, the one or more TET enzymes comprise a TET2 enzyme comprising a T1372S mutation, such as TET2-CS-T1372S and TET2-CD-T1372S. Examples of TET2-CS-T1372S and TET2-CD-T1372S are provided as SEQ ID NOs: 11 and 12. A TET2 comprising a T1372S mutation is described in U.S. Pat. No. 10,961,525. TET2 may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525 (SEQ ID NO: 1 of the present application). Thus, the sequence of a T1372S TET2 catalytic domain may be obtained by changing the threonine at position 258 of SEQ ID NO: 21 of U.S. Pat. No. 10,961,525 to serine. T1372S TET2 is also described in Liu et al., Nat Chem Biol. 2017 February; 13 (2): 181-187. Although Liu et al. states that the T1372S mutant “exhibits WT-like activity,” it is recognized herein that per results in Liu et al., a TET2 comprising a T1372S mutation can more efficiently oxidize 5mC to produce 5-carboxylcytosine (5caC) (as opposed to other, less oxidized intermediaries, e.g., 5hmC and 5fC) than other versions of TET2 such as TET2 lacking a T1372S mutation. In some embodiments, the TET2 enzyme comprises SEQ ID NO: 14 or optionally a variant of SEQ ID NO: 14 in which at least 5, 6, 7, or 8 positions match SEQ ID NO: 14 including position 5 of SEQ ID NO: 14. In some embodiments, the TET2 enzyme is a human TET2 enzyme comprising a T1372S mutation. In some embodiments, the TET2 enzyme comprises the sequence of SEQ ID NO: 11. In some embodiments, the TET2 enzyme comprises a sequence having at least 80%, 85%, 90%, 95%, 96%, 97%, 98%, or 99% identity to SEQ ID NO: 11. In some embodiments, the TET2 enzyme comprises a sequence having at least 80%, 85%, 90%, 95%, 96%, 97%, 98%, or 99% identity to SEQ ID NO: 12. In some embodiments, the TET2 enzyme comprises the sequence of SEQ ID NO: 12. The sequences of SEQ ID NOs: 11 and 12 are shown in the Table of Sequences herein.

In some embodiments, the one or more TET enzymes comprise a V1900 TET mutant, such as a V1900A, V1900C, V1900G, V1900I, or V1900P TET mutant. In some embodiments, the one or more TET enzymes comprise a V1900 TET2 mutant, such as a V1900A, V1900C, V1900G, V1900I, or V1900P TET2 mutant. Examples of V1900A, V1900C, V1900G, V1900I, and V1900P TET2 mutants are provided as SEQ ID NOs: 2-6. While Liu et al. states that “the Val 1900 position is fairly tolerant to mutation, with a variety of mutants showing WT-like stepwise oxidation or reduced overall activity,” it is recognized herein that per results in Liu et al., in some embodiments, these mutants can more efficiently oxidize 5mC to produce 5-carboxylcytosine (5caC) (as opposed to other, less oxidized intermediaries, e.g., 5hmC and 5fC) than other versions of TET2 lacking one of these mutations. In some embodiments, the V1900 TET mutant has at least 80%, 85%, 90%, 95%, 96%, 97%, 98%, or 99% sequence identity to SEQ ID NO: 2, 3, 4, 5, or 6. Position 1900 of the wild-type TET2 sequence corresponds to position 438 in each of SEQ ID NOs: 1-6. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Because 5-caC is significantly less likely to be erroneously deaminated (e.g., by a cytosine deaminase, such as by an APOBEC enzyme) than less oxidized modified cytosines (such as 5-fC), maximizing formation of 5-caC (and, e.g., reducing the amount of 5-fC in the DNA) reduces the risk of false calls in which a base that was methylated (or hydroxymethylated) in the original DNA sample is identified as unmethylated because it was erroneously deaminated (e.g., even though it had been oxidized to 5-fC). Accordingly, in some embodiments, the TET enzyme comprises a mutation that increases formation of 5-caC. Exemplary mutations (such as T1372S, V1900A, V1900C, V1900G, V1900I, or V1900P TET) are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions).

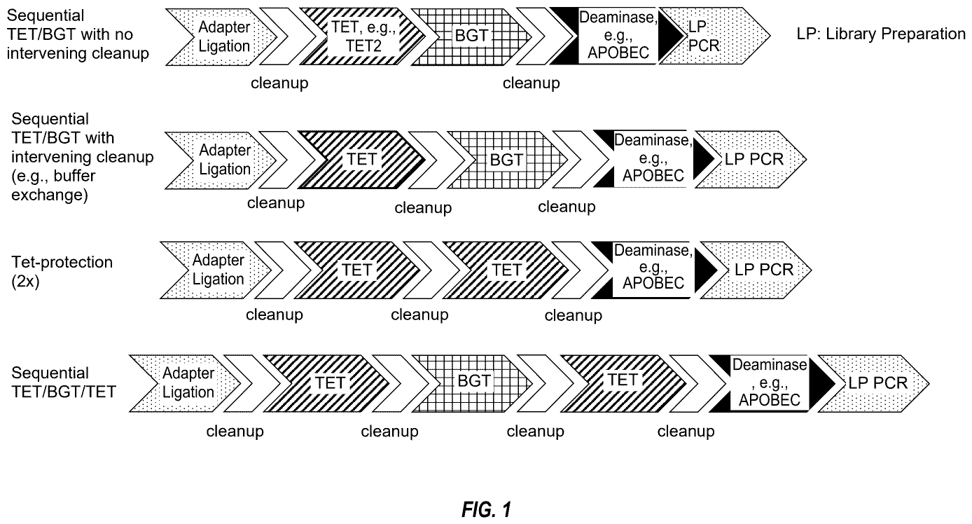

Also provided herein is a method comprising a) contacting DNA with one or more TET enzymes to oxidize 5-methylcytosine (5mC) present in the DNA, wherein, for at least a portion of the 5mC, the oxidation of 5mC results in formation of 5-carboxylcytosine (5-caC), wherein the one or more TET enzymes comprises a V1900A mutant TET or a V1900P mutant TET, or wherein at least one of the TET enzymes comprises a mutation that increases formation of 5-caC; after a), contacting at least a portion of the DNA with a cytosine deaminase, thereby converting unmethylated cytosine in the DNA to uracil, thereby producing treated DNA; and sequencing at least a portion of the treated DNA. A V1900A mutant TET and a V1900P mutant TET may possess sufficient enzymatic activity to convert 5-methylated cytosine (5mC) to 5-carboxycytosine (5caC) in a substantially complete manner, such that further treatment (e.g., with βGT or other reagents for further modifying 5hmC and/or 5fC) may not be required, as recognized herein; see discussion above regarding Supplementary FIG. 1 of Liu et al., Nat Chem Biol. 2017 February; 13 (2): 181-18. Accordingly, in some embodiments, the method does not comprise contacting the DNA with a glucosyltransferase, e.g., concurrently with or after being contacted with the one or more TET enzymes. In some embodiments, the method does not comprise contacting the DNA with an alkoxylamine, such as a C₂₋₆ alkoxylamine, such as ethoxylamine (EtONH₂), e.g., concurrently with or after being contacted with the one or more TET enzymes. In some embodiments, the method does not comprise contacting the DNA with a ruthenate such as KRuO₄, e.g., concurrently with or after being contacted with the one or more TET enzymes. In some embodiments, the method does not comprise contacting the DNA with a reducing agent such as NaBH₄, e.g., concurrently with or after being contacted with the one or more TET enzymes. In some embodiments, the method does not comprise contacting the DNA with Pinnick oxidation reagents, e.g., concurrently with or after being contacted with the one or more TET enzymes. In some embodiments, the method does not comprise contacting the DNA with 4-acetamido-2,2,6,6-tetramethylpiperidine-1-oxoammonium tetrafluoroborate (ACT⁺BF₄⁻), e.g., concurrently with or after being contacted with the one or more TET enzymes. In some embodiments, the DNA is not subjected to a further treatment that reduces the amount of 5fC in the DNA and/or protects 5-hydroxymethylcytosine that may be present in the DNA, e.g., concurrently with or after being contacted with the one or more TET enzymes. Here too, using a TET enzyme that maximizes formation of 5-caC can be beneficial relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample.

In some embodiments, the DNA is contacted with the one or more TET enzymes in the absence of βGT. It can be helpful to perform these steps separately in that the reactions can be run at different pHs, as TET enzymes may perform better at slightly acidic to neutral pH (e.g., 6-7 such as 6.5-6.8 or other exemplary ranges or values described elsewhere herein), while βGT may perform better at slightly alkaline pH (e.g., 7.5-8.5 or about 8).

In some embodiments, the DNA is contacted with βGT after it was contacted with the one or more TET enzymes.

In some embodiments, the further treatment comprises contacting the DNA with β-glucosyltransferase (βGT) or by carbamoylation of the 5hmCs (e.g., using 5-hydroxymethylcytosine carbamoyltransferase). βGT is also referred to herein as βGT. An exemplary βGT is T4 βGT. In this method, 5hmC can be protected from conversion, for example through glucosylation using β-glucosyl transferase (βGT), forming (5-glucosylhydroxymethylcytosine) 5ghmC, or through carbamoylation using 5-hydroxymethylcytosine carbamoyltransferase, forming 5cmC. Examples thereof are described, for example, in Yu et al., Cell 2012; 149:1368-80, and in Yang et al., Bio-protocol, 2023; 12 (17): e4496. As described elsewhere herein, certain APOBEC enzymes can deaminate 5hmC in DNA, although 5hmC deamination typically occurs at lower rates than that of 5mC, see, e.g., Schutsky et al., Nature Biotechnology, 2018; 36:1083-1090; and Schutsky et al., Nucleic Acids Research, 2017; 45 (13): 7655-7665. Glucosylation or carbamoylation of 5hmC can reduce or eliminate deamination of 5hmC by a deaminase, such as APOBEC3A (which preferentially deaminates unmethylated C and 5mC, but has low but detectable activity on 5hmC and 5fC). βGT enzymes are commercially available, e.g., from New England Biolabs or Cambridge Epigenetix. The use of βGT is described, e.g., in Vaisvila et al., Genome Res. 2021; 31:1280-1289, Füllgrabe, J. et al., Nat. Biotechnol. https://doi.org/10.1038/s41587-022-01652-0 (2023), and in WO2022/023753 A1.

In some embodiments, the further treatment comprises contacting the DNA with β-glucosyltransferase (βGT) or the 5-hydroxymethylcytosine carbamoyltransferase and then contacting the DNA with one or more TET enzymes to further oxidize the 5fC.

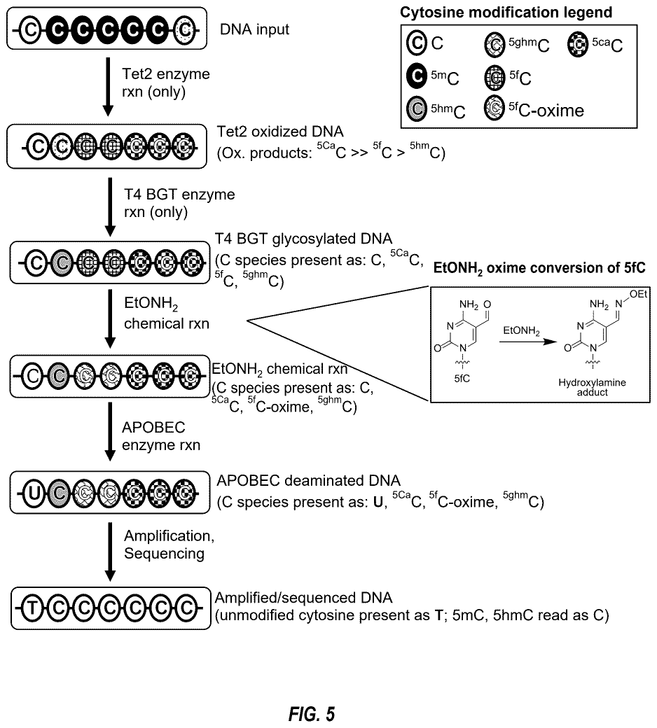

In some embodiments, the further treatment comprises contacting the DNA with βGT or 5-hydroxymethylcytosine carbamoyltransferase and then contacting the DNA with an alkoxylamine, such as a C₂₋₆ alkoxylamine, such as ethoxylamine (EtONH₂). Exemplary reaction conditions for treating DNA with an alkoxylamine such as EtONH₂ are described in Song et al., Cell 2013 Apr. 25; 153 (3): 678-691. As EtONH₂ treatment is generally mild and robust, it may be added directly to enzyme buffers without an intervening cleanup after the preceding step. Treatment with ethoxylamine converts 5fC to an adduct comprising an oxime moiety, referred to as 5fC-oxime. See FIG. 5 for the structure of the hydroxylamine adduct referred to as 5fC-oxime.

In some embodiments, the further treatment comprises contacting the DNA with a ruthenate such as KRuO₄ and then contacting the DNA with an alkoxylamine, such as a C₂₋₆ alkoxylamine, such as EtONH₂. Treatment with a ruthenate such as KRuO4 can oxidize 5hmC to 5fC.

In some embodiments, the further treatment comprises contacting the DNA with a reducing agent such as NaBH₄ and then contacting the DNA with βGT or 5-hydroxymethylcytosine carbamoyltransferase. NaBH₄ reduces 5fC to 5hmC. See Booth et al., Nat Chem. 2014 May; 6 (5): 435-440.

In some embodiments, the further treatment comprises contacting the DNA with βGT or 5-hydroxymethylcytosine carbamoyltransferase and then contacting the DNA with Pinnick oxidation reagents. In some embodiments, the further treatment comprises contacting the DNA with a ruthenate such as KRuO₄ and then contacting the DNA with Pinnick oxidation reagents. In some embodiments, the further treatment comprises contacting the DNA with 4-acetamido-2,2,6,6-tetramethylpiperidine-1-oxoammonium tetrafluoroborate (ACT₊BF₄⁻) and then contacting the DNA with Pinnick oxidation reagents. In some embodiments, the Pinnick oxidation reagents comprise a chlorite salt and an alkene. In some embodiments, the chlorite salt is sodium chlorite and/or the alkene is a branched C₅₋₁₀ alkene, such as a branched C₅₋₇ alkene, such as 2-methyl-2-butene. Pinnick oxidation reagents and their use in oxidizing 5fC to 5caC are described in Xu et al., J. Am. Chem. Soc. 2023; 145:7095-7100. 4-acetamido-2,2,6,6-tetramethylpiperidine-1-oxoammonium tetrafluoroborate (ACT⁺BF₄⁻) and its use in oxidizing 5hmC to 5fC are also described in Xu et al., J. Am. Chem. Soc. 2023; 145:7095-7100.

In some embodiments, the further treatment comprises contacting the DNA with 4-acetamido-2,2,6,6-tetramethylpiperidine-1-oxoammonium tetrafluoroborate (ACT⁺BF₄⁻) and then contacting the DNA with an alkoxylamine, such as a C₂₋₆ alkoxylamine, such as EtONH₂.

In some embodiments, the further treatment comprises contacting the DNA with one or more TET enzymes a second time after step a), wherein the method further comprises purifying the DNA between steps a) and b), optionally wherein the DNA is purified using solid phase reversible immobilization (SPRI).

In some embodiments, the DNA is contacted with the one or more TET enzymes in a buffer having a pH of about 6.5, 6.6, 6.7, or 6.8, or a pH of 6.65+x where x is 0.7, 0.6, 0.5, 0.4, 0.3, 0.2, or 0.15. In some embodiments, the DNA is contacted with the one or more TET enzymes in a buffer having a pH ranging from 6 to 7. In some embodiments, the DNA is contacted with the one or more TET enzymes in a buffer having a pH ranging from 6.4 to 6.9. In some embodiments, the DNA is contacted with the one or more TET enzymes in a buffer having a pH ranging from 6.5 to 6.8.

In some embodiments, the cytosine deaminase is an AID/APOBEC family cytosine deaminase enzyme, such as an APOBEC enzyme, optionally wherein the APOBEC enzyme is APOBEC3A. Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. Of the APOBEC family members, APOBEC3A has high activity and a preference for 5mC deamination. For an exemplary description of APOBEC conversion, see, e.g., Schutsky et al., Nature Biotechnology 2018; 36:1083-1090. In Schutsky et al., Nucleic Acids Research, 2017; 45 (13): 7655-7665, A3A deaminated 5hmC at an approximately 5600-fold slower rate than unmodified cytosine, and deaminated 5fC at an approximately 3700-fold slower rate than unmodified cytosine. In the same study, A3A had no detectable deaminase activity on 5caC. Accordingly, in some embodiments of the disclosed methods, because A3A has some (low but detectable) activity on 5hmC and 5fC, but not on 5caC, it can be beneficial to use a TET enzyme that maximizes formation of 5-caC relative to less oxidized modified cytosines (e.g., 5hmC and 5caC).

In some embodiments, the APOBEC enzyme is HYPER-A3B-1. HYPER-A3B-1 is described in U.S. Pat. No. 10,961,525 and its sequence appears as SEQ ID NO: 8 therein (SEQ ID NO: 15 herein).

Contacting the DNA with a cytidine deaminase may be referred to herein as a conversion step.

In some embodiments, the conversion procedure comprises enzymatic conversion of unmodified nucleosides, such as unmodified cytosines using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of the first nucleobase using MsddA. In some embodiments, the procedure that affects a first nucleobase in the DNA differently from a second nucleobase in the DNA comprises enzymatic conversion of the first nucleobase using the methyl-sensitive deaminase, AshDa01, which is described in WO 2023/097226 A2. Optionally, however, in some embodiments of the disclosed methods wherein the conversion procedure deaminates unmodified nucleosides (such as unmodified cytosines), the method further comprises enzymatic protection of at least one type of modified nucleoside (such as modified cytosines, such as 5mC and/or 5hmC) in the DNA prior to deamination of (unprotected) unmodified nucleosides (such as unmodified cytosines). In some embodiments, the at least one type of modified nucleoside is 5mC. In some embodiments, enzymatic protection of 5mC comprises converting a 5mC to carboxylcytosine. For example, converting a 5mC to carboxylcytosine can comprise contacting the 5mC with a TET enzyme, such as TET1, TET2, or TET3, or any suitable TET enzyme disclosed herein (such as a a TETv, a TETcd, or a TET comprising a mutation such as T1372S, V1900A, V1900C, V1900I, V1900P, V1900S, or V1900T). In some embodiments, the at least one type of modified nucleoside is 5hmC. In some embodiments, the enzymatic protection of 5hmCs in the DNA prior to the deamination of unmodified cytosines comprises glucosylation of the 5hmCs, such as described herein.

In some embodiments, the DNA comprises a first member of a binding pair, optionally wherein the first member of the binding pair is biotin. An exemplary second member of the binding pair is streptavidin. Other suitable binding pairs are known in the art.

In some embodiments, the first member of the binding pair is attached to an adapter joined to the DNA.

The method of claim 28 or 29, wherein the DNA is bound to a solid phase comprising a second member of the binding pair during steps a)-b), optionally wherein the solid phase comprises beads and/or the second member of the binding pair is streptavidin.

In some embodiments, the DNA is released from the solid phase before step c).

In some embodiments, the DNA is released from the solid phase by cleaving a cleavable moiety in an adapter joined to the DNA, optionally wherein the cleavable moiety comprises uracil or photocleavable biotin.

In some embodiments, the method further comprises enriching the DNA by capturing a target region set from the sample, wherein the capture step is before, after or in between step (a) and step (c) of Embodiment 1 or any dependent embodiment thereof.

In some embodiments, the method further comprises comparing the sequence data obtained with a pre-determined reference sequence; and/or sequence data obtained by sequencing a sub-sample of the DNA that was not subjected to the conversion procedure; and identifying point differences between the converted DNA sequences and the reference sequence (A) or non-converted DNA sequence data (B) as nucleosides having a modification status that permits a change in base pairing specificity on exposure to the cytosine deaminase. In a particular embodiment, the method further comprises comparing the sequence data obtained with sequence data obtained by sequencing a sub-sample of the DNA that was not subjected to the conversion procedure; and identifying point differences between the converted DNA sequences and the non-converted DNA sequence data as nucleosides having a modification status that permits a change in base pairing specificity on exposure to the cytosine deaminase. In another particular embodiment, the method further comprises comparing the sequence data obtained with a pre-determined reference sequence; and identifying point differences between the converted DNA sequences and the reference sequence as nucleosides having a modification status that permits a change in base pairing specificity on exposure to the cytosine deaminase.

In some embodiments, the DNA comprises cell-free DNA (cfDNA), optionally cfDNA obtained from a test subject, optionally wherein the test subject is a patient having or suspected of having cancer.

In some embodiments, the method further comprises using the detection of modified nucleosides in the DNA sample to determine or predict the presence of DNA produced by a cancer cell or tumor, to determine the probability that a test subject has a tumor or cancer, or to characterize a cancer or tumor of the subject.

In some embodiments, a non-converted subsample of the DNA is not contacted with the cytosine deaminase before sequencing, wherein the treated subsample and the non-converted subsample have different adapter sequences, and wherein the converted subsample and the non-converted subsample are recombined before sequencing step (d).

In some embodiments, the method further comprises analyzing the DNA to detect copy number variation, single nucleotide variants, insertions, deletions, methylation, and/or fusions.

In some embodiments, the method further comprises analyzing the DNA to detect methylation and one or more of copy number variation, single nucleotide variants, insertions, deletions, and/or fusions.

In some embodiments, the method further comprises capturing epigenetic target regions from the adapter-ligated DNA and amplifying and sequencing the epigenetic target regions.

In some embodiments, the captured epigenetic target regions form an epigenetic target region set.

In some embodiments, the epigenetic target region set comprises a plurality of type-specific epigenetic target regions, and wherein the type-specific epigenetic target regions are type-specific differentially methylated regions and/or type specific fragments.

In some embodiments, wherein the plurality of type-specific epigenetic target regions comprises type-specific hypomethylated regions.

In some embodiments, the sample is a blood sample.

In some embodiments, wherein the plurality of type-specific epigenetic target regions comprises target regions that are: hypermethylated in immune cells relative to non-immune cell types present in the blood sample; differentially methylated in colon relative to other tissue types; differentially methylated in breast relative to other tissue types; differentially methylated in liver relative to other tissue types; differentially methylated in kidney relative to other tissue types; differentially methylated in pancreas relative to other tissue types; differentially methylated in prostate relative to other tissue types; differentially methylated in skin relative to other tissue types; or differentially methylated in bladder relative to other tissue types.

In some embodiments, the plurality of type-specific epigenetic target regions comprises:
target regions that are hypomethylated in non-immune blood cells relative to the methylation level of the target regions in a different cell or tissue type in the sample; fragments specific to immune cells relative to non-immune cell types present in the blood sample; or fragments specific to colon, lung, breast, liver, kidney, pancreas, prostate, skin, or bladder relative to other tissue types.

In some embodiments, the method further comprises identifying at least one cell type or tissue type from which the type-specific epigenetic target regions originated.

In some embodiments, the level of type-specific epigenetic target regions that originated from a cell or tissue type is determined.

In some embodiments, the levels of type-specific epigenetic target regions that originated from immune cells, non-immune blood cells, colon, lung, breast, liver, kidney, prostate, skin, bladder, or pancreas are determined. In some embodiments, the type-specific epigenetic target regions comprise cell-type specific, tissue-type specific, and/or cancer-type specific epigenetic target regions.

In some embodiments, the blood sample is fractionated prior to capturing at least an epigenetic target region set of DNA.

In some embodiments, the method further comprises detecting the presence or absence of sequence variations and/or determining fragmentation patterns, wherein adapted DNA comprising quality control nucleosides indicative of sub-optimal or erroneous conversion of quality control nucleosides is included in detecting the presence or absence of sequence variations and/or determining fragmentation patterns.

In some embodiments, oligonucleotide adapters are ligated to the DNA. In some embodiments, the oligonucleotide adapters are ligated to the DNA before step a) of Embodiment 1 or any dependent embodiment thereof.

In some embodiments, the oligonucleotide adapters comprise sequencing primer binding sites.

In some embodiments, the method further comprises amplifying the DNA using primers targeting the adapters, wherein the amplifying step is between step (c) and the sequencing step (d) of Embodiment 1 or any dependent embodiment thereof.

In some embodiments, one or more nucleosides of the adapter is a modified nucleoside, optionally wherein the modified nucleoside comprises a modified cytosine. The modified cytosine may be deamination-resistant. In some embodiments, the modified nucleoside is 5-carboxyl cytosine, pyrrolo cytosine, or 5-propynyl cytosine. For an exemplary description of adapters and deamination-resistant modified cytosines, see WO2023/288222A1.

The method may further comprise enriching the DNA by capturing a target region set from the sample. The capture step may be before, after or in between ligating adapters (if performed) or step (a) and the conversion step (c) of Embodiment 1 or any dependent embodiment thereof.

Sequencing step (d) allows modified nucleosides in the initial sample to be identified as those that have been converted. For example, the method may comprise: (i) comparing the sequence data obtained in step (d) of Embodiment 1 or any dependent embodiment thereof with (A) a (pre-determined) reference sequence(s); and/or (B) sequence data obtained by sequencing a sub-sample of the DNA that was not subjected to the conversion procedure; and (ii) identifying point differences between the converted DNA sequences and the reference sequence (A) or non-converted DNA sequence data (B) as nucleotides having a modification status that permits a change in base pairing specificity on exposure to the conversion procedure. By way of another example, the method may comprise: (i) comparing the sequence data obtained in step (d) of Embodiment 1 or any dependent embodiment thereof with a (pre-determined) reference sequence(s); and (ii) identifying point differences between the converted DNA sequences and the reference sequence as nucleotides having a modification status that permits a change in base pairing specificity on exposure to the conversion procedure. By way of another example, the method may comprise: (i) comparing the sequence data obtained in step (d) of Embodiment 1 or any dependent embodiment thereof with sequence data obtained by sequencing a sub-sample of the DNA that was not subjected to the conversion procedure; and (ii) identifying point differences between the converted DNA sequences and the or non-converted DNA sequence data as nucleotides having a modification status that permits a change in base pairing specificity on exposure to the conversion procedure.

In some embodiments, the DNA comprises cell-free DNA (cfDNA). The cfDNA may, for example, be obtained from a test subject. In some cases, the test subject is a patient having or suspected of having cancer.

In some cases, the method may further comprise using the detection of modified nucleosides in the DNA sample to determine or predict the presence of DNA produced by a cancer cell or tumor, to determine the probability that a test subject has a tumor or cancer, or to characterize a cancer or tumor of the subject.

In some embodiments, a sub-sample of the DNA is not subjected to the conversion procedure before sequencing. In some cases, the converted subsample and the non-converted subsample may have different adapter sequences. The converted subsample and the non-converted subsample may be recombined for sequencing step (c). The different adapter sequences may be used to distinguish sequences or molecules from the exposed subsample and the non-exposed subsample in later steps or analysis.

In a further aspect, the disclosure provides a method of detecting modified nucleosides in a DNA sample. The method may comprise any set of steps (including but not limited to (a) to (d)) set out above. Other features set out above, where appropriate, are also applicable to such methods. For example, the method may further comprise comparing the sequence data obtained in step (d) with (A) pre-determined reference sequences; and/or (B) sequence data obtained by sequencing a sub-sample of the DNA that was not subjected to the conversion procedure; and identifying point differences between the converted DNA sequences and the reference or non-converted DNA sequences as modified nucleotides in the DNA sample.

In some embodiments, the results of the methods disclosed herein are used as an input to generate a report. The report may be in a paper or electronic format. For example, the detection of false positives and/or false negatives, as obtained by the methods disclosed herein, or information derived therefrom, can be displayed directly in such a report. Alternatively, or additionally, diagnostic information or therapeutic recommendations which are at least in part based on the methods disclosed herein can be included in the report.

The various steps of the methods disclosed herein may be carried out at the same or different times, in the same or different geographical locations, e.g., countries, and/or by the same or different people.

In a particular embodiment, the method comprises contacting DNA with a TET enzyme (such as a TET1 or TET2 enzyme); subjecting the DNA to a further treatment comprising contacting the DNA with β-glucosyltransferase (βGT); contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with β-glucosyltransferase (βGT); contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with β-glucosyltransferase (βGT); contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with β-glucosyltransferase (βGT) and then contacting the DNA with one or more TET enzymes to further oxidize the 5fC; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with β-glucosyltransferase (βGT) and then contacting the DNA with one or more TET enzymes to further oxidize the 5fC; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with β-glucosyltransferase (βGT) and then contacting the DNA with one or more TET enzymes to further oxidize the 5fC; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with βGT and then contacting the DNA with an alkoxylamine, such as a C₂₋₆ alkoxylamine, such as ethoxylamine (EtONH₂); contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with βGT and then contacting the DNA with an alkoxylamine, such as a C₂₋₆ alkoxylamine, such as ethoxylamine (EtONH₂); contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with βGT and then contacting the DNA with an alkoxylamine, such as a C₂₋₆ alkoxylamine, such as ethoxylamine (EtONH₂); contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with a ruthenate such as KRuO₄ and then contacting the DNA with an alkoxylamine, such as a C₂₋₆ alkoxylamine, such as EtONH₂; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with a ruthenate such as KRuO₄ and then contacting the DNA with an alkoxylamine, such as a C₂₋₆ alkoxylamine, such as EtONH₂; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with a ruthenate such as KRuO₄ and then contacting the DNA with an alkoxylamine, such as a C₂₋₆ alkoxylamine, such as EtONH₂; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with a reducing agent such as NaBH₄ and then contacting the DNA with βGT; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with a reducing agent such as NaBH₄ and then contacting the DNA with βGT; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with a reducing agent such as NaBH₄ and then contacting the DNA with βGT; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with βGT and then contacting the DNA with Pinnick oxidation reagents; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with βGT and then contacting the DNA with Pinnick oxidation reagents; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with βGT and then contacting the DNA with Pinnick oxidation reagents; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with a ruthenate such as KRuO₄ and then contacting the DNA with Pinnick oxidation reagents; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with a ruthenate such as KRuO₄ and then contacting the DNA with Pinnick oxidation reagents; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with a ruthenate such as KRuO₄ and then contacting the DNA with Pinnick oxidation reagents; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with 4-acetamido-2,2,6,6-tetramethylpiperidine-1-oxoammonium tetrafluoroborate (ACT⁺BF₄⁻) and then contacting the DNA with Pinnick oxidation reagents; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with 4-acetamido-2,2,6,6-tetramethylpiperidine-1-oxoammonium tetrafluoroborate (ACT⁺BF₄⁻) and then contacting the DNA with Pinnick oxidation reagents; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with 4-acetamido-2,2,6,6-tetramethylpiperidine-1-oxoammonium tetrafluoroborate (ACT⁺BF₄⁻) and then contacting the DNA with Pinnick oxidation reagents; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with 4-acetamido-2,2,6,6-tetramethylpiperidine-1-oxoammonium tetrafluoroborate (ACT⁺BF₄⁻) and then contacting the DNA with an alkoxylamine, such as a C₂₋₆ alkoxylamine, such as EtONH₂; contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with 4-acetamido-2,2,6,6-tetramethylpiperidine-1-oxoammonium tetrafluoroborate (ACT⁺BF₄⁻) and then contacting the DNA with an alkoxylamine, such as a C₂₋₆ alkoxylamine, such as EtONH₂; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with 4-acetamido-2,2,6,6-tetramethylpiperidine-1-oxoammonium tetrafluoroborate (ACT⁺BF₄⁻) and then contacting the DNA with an alkoxylamine, such as a C₂₋₆ alkoxylamine, such as EtONH₂; contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET enzyme (such as a TET1 or TET2 enzyme); subjecting the DNA to a further treatment comprising contacting the DNA with one or more TET enzymes a second time after step a), wherein the method further comprises purifying the DNA between steps a) and b), optionally wherein the DNA is purified using solid phase reversible immobilization (SPRI); contacting at least a portion of the DNA with an APOBEC enzyme (such as APOBEC3A), thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET1 enzyme; subjecting the DNA to a further treatment comprising contacting the DNA with one or more TET enzymes a second time after step a), wherein the method further comprises purifying the DNA between steps a) and b), optionally wherein the DNA is purified using solid phase reversible immobilization (SPRI); contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

In a particular embodiment, the method comprises contacting DNA with a TET2 enzyme; subjecting the DNA to a further treatment comprising contacting the DNA with one or more TET enzymes a second time after step a), wherein the method further comprises purifying the DNA between steps a) and b), optionally wherein the DNA is purified using solid phase reversible immobilization (SPRI); contacting at least a portion of the DNA with an APOBEC3A, thereby converting unmethylated cytosine in the DNA to uracil, and producing treated DNA; and sequencing at least a portion of the treated DNA, such as to detect one or more modified nucleosides in the DNA. In some such embodiments, the DNA is cfDNA. In some embodiments, the method further comprises using the detection of modified nucleosides in the DNA sample (such as the cfDNA sample) to determine or predict the presence of DNA (such as cfDNA) produced by a cancer cell or tumor, to determine the probability that a test subject has a tumor or cancer, or to characterize a cancer or tumor of the subject.

### Samples and Subjects

The disclosure relates to methods of analyzing the modified nucleoside profile of DNA in a sample. In some cases, the nucleic acid is obtained or has been obtained from a subject. In some embodiments, the nucleic acid sample may comprise or consist of nucleic acid, e.g., DNA, from a biological sample obtained from a subject. The subject may be a human, a mammal, an animal, a primate, rodent (including mice and rats), or other common laboratory, domestic, companion, service or agricultural animal, for example a rabbit, dog, cat, horse, cow, sheep, goat or pig. The subject may in some cases have or be suspected of having a cancer, tumor or neoplasm. In other cases, the subject may not have cancer or a detectable cancer symptom. The subject may have been treated with one or more cancer therapy, e.g., any one or more of chemotherapies, antibodies, vaccines or biologics. The subject may be in remission, e.g. from a tumor, cancer, or neoplasia (e.g., following treatment such as chemotherapy, surgical resection, radiation, or a combination thereof). The subject may or may not be diagnosed as being susceptible to cancer or any cancer-associated genetic mutations/disorders. In some embodiments, the sample is a polynucleotide sample obtained from a tumor tissue biopsy. The cancer, tumor or neoplasm may generally be of any type, for example a cancer tumor or neoplasm of the lung, colon, rectum (or colorectum), kidney, breast, prostate, or liver, or other type of cancer as described herein. In some embodiments, the sample is obtained from a subject in remission from a tumor, cancer, or neoplasia (e.g., following chemotherapy, surgical resection, radiation, or a combination thereof). In any of the foregoing embodiments, the pre-cancer, cancer, tumor, or neoplasia or suspected pre-cancer, cancer, tumor, or neoplasia may be of the bladder, head and neck, lung, colon, rectum, kidney, breast, prostate, skin, or liver. In some embodiments, the pre-cancer, cancer, tumor, or neoplasia or suspected pre-cancer, cancer, tumor, or neoplasia is of the lung. In some embodiments, the pre-cancer, cancer, tumor, or neoplasia or suspected pre-cancer, cancer, tumor, or neoplasia is of the colon or rectum. In some embodiments, the pre-cancer, cancer, tumor, or neoplasia or suspected pre-cancer, cancer, tumor, or neoplasia is of the breast. In some embodiments, the pre-cancer, cancer, tumor, or neoplasia or suspected pre-cancer, cancer, tumor, or neoplasia is of the prostate. In any of the foregoing embodiments, the subject may be a human subject. In some embodiments, the sample is obtained from a subject having a stage I cancer, stage II cancer, stage III cancer or stage IV cancer.

The sample can be any biological sample isolated from a subject. The sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another.

In some embodiments, a population of nucleic acids is obtained from a serum, plasma or blood sample from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications. Post-replication modifications include modifications of cytosine, particularly at the 5-position of the nucleobase, e.g., 5-methylcytosine, 5-hydroxymethylcytosine, 5-formylcytosine and 5-carboxylcytosine.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines or biologies. The subject may be in remission. The subject may or may not be diagnosed of being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For example, the volume can be 0.5 mL, 1 mL, 5 mL 10 mL, 20 mL, 30 mL, or 40 mL. A volume of sampled plasma may be 5 to 20 mL.

A sample can comprise various amounts of nucleic acid that contains genome equivalents. For example, a sample of about 30 ng DNA can contain about 10,000 (10⁴) haploid human genome equivalents and, in the case of cell free DNA (cfDNA), about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

The sample may be or comprise cell free nucleic acids or cfDNA. The cfDNA may be obtained from a test subject, for example as described above. For example, the sample for analysis may be plasma or serum containing cell-free nucleic acids. “Cell-free DNA” “cfDNA molecules,” or “cfDNA”, for example, include DNA molecules that naturally occur in a subject in extracellular form (e.g., in blood, serum, plasma, or other bodily fluids such as lymph, cerebrospinal fluid, urine, or sputum). While the cfDNA originally existed in a cell or cells in a large complex biological organism, e.g., a mammal, it has undergone release from the cell(s) in vivo into a fluid found in the organism, and may be obtained by obtaining a sample of the fluid without the need to perform an in vitro cell lysis step. In other words, cell-free nucleic acids or DNA are nucleic acids or DNA not contained within or otherwise bound to a cell, or the nucleic acids or DNA remaining in a sample after removing intact cells. Cell-free nucleic acids include DNA, RNA, and hybrids thereof, including genomic DNA, mitochondrial DNA, siRNA, miRNA, circulating RNA (cRNA), tRNA, IRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA). In some embodiments, cell free nucleic acids are produced by tumor cells. In some embodiments, cell free nucleic acids are produced by a mixture of tumor cells and non-tumor cells.

Exemplary amounts of cell-free nucleic acids in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 μg, at least 100 μg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 μg, 1 ng, 10 ng, 100 ng, 150 ng, or 200 ng of cell-free nucleic acid molecules. The method can comprise obtaining 1 femtogram (fg) to 200 ng of cell-free nucleic acid molecules from samples.

Cell-free DNA refers to DNA not contained within a cell at the time of its isolation from a subject. For example, cfDNA can be isolated from a sample as the DNA remaining in the sample after removing intact cells, without lysing the cells or otherwise extracting intracellular DNA. Cell-free nucleic acids include DNA, RNA, and hybrids thereof, including genomic DNA, mitochondrial DNA, siRNA, miRNA, circulating RNA (cRNA), tRNA, IRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides.

Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with an alcohol. Further clean up steps may be used such as silica-based columns to remove contaminants or salts. Non-specific bulk carrier nucleic acids, such as C 1 DNA, DNA or protein for hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

Double-stranded DNA molecules in a sample and single stranded nucleic acid molecules converted to double stranded DNA molecules can be linked to adapters at either one end or both ends. Typically, double stranded molecules are blunt ended by treatment with a polymerase with a 5′-3′ polymerase and a 3 ‘-5’ exonuclease (or proof reading function), in the presence of all four standard nucleotides. Klenow large fragment and T4 polymerase are examples of suitable polymerase. The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### Ligation to Adapters

In some embodiments, the methods comprise ligating adapters to DNA. Double-stranded nucleic acids e.g., DNA molecules in a sample, and single stranded nucleic acid molecules converted to double stranded molecules, can be linked to adapters at either one end or both ends. In some cases, the DNA is made ligatable, e.g., by extending the end overhangs of the DNA molecules, and adding adenosine residues to the 3′ ends of fragments and phosphorylating the 5′ end of each DNA fragment. Typically, double stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase.

The blunt ended DNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample nucleic acid molecule. In some instances, two adapters can be ligated to a single sample nucleic acid molecule, with one adapter ligated to each end of the sample nucleic acid molecule.

In some embodiments, the ligase used in ligation reactions can act on both single strand DNA nicks and double stranded DNA ends. In some cases, the ligase is T4 DNA ligase or T3 DNA ligase. Adapters can include nucleic acid primer binding sites to permit amplification of a sample nucleic acid molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given nucleic acid molecule. Adapters of the same or different sequence can be linked to the respective ends of a sample nucleic acid molecule. In some cases, adapters of the same or different sequence are linked to the respective ends of the nucleic acid molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a nucleic acid molecule to be analyzed. Other exemplary adapters include T-tailed, C-tailed or hairpin shaped adapters. For example, a hairpin shaped adaptor can comprise a complementary double stranded portion and a loop portion, where the double stranded portion can be attached (e.g., ligated) to a double-stranded polynucleotide. Hairpin shaped sequencing adaptors can be attached to both ends of a polynucleotide fragment to generate a circular molecule, which can be sequenced multiple times. The adapters used in the methods of the present disclosure comprise one or more known modified nucleosides. In some embodiments, the one or more known modified nucleosides include enzymatic deamination-resistant nucleosides, e.g., enzymatic deamination-resistant cytosines, such as 5-carboxyl cytosine, 5-carboxymethyl cytosine, pyrrolo cytosine, or a 5-(C₂₋₆ alkynyl) cytosine such as 5-propynyl cytosine. In instances where two adapters are ligated to a sample nucleic acid (one at each end), either or both of the adapters may comprise one or more known modified nucleosides. Typically, the primer binding site(s), sequencing primer binding site(s), sample index(es) and/or molecular barcode(s), if present, do not comprise the known modified nucleosides that change base pairing specificity as a result of the conversion procedure, but may comprise one or more other known modified nucleosides (such as one or more enzymatic deamination-resistant nucleosides, e.g., enzymatic deamination-resistant cytosines, such as 5-carboxyl cytosine, 5-carboxymethyl cytosine, pyrrolo cytosine, or a 5-(C₂₋₆ alkynyl) cytosine such as 5-propynyl cytosine).

In some embodiments, sample nucleic acids flanked by adapters can be amplified by PCR and other amplification methods. Such amplification can be used to increase the amount of DNA available for subsequent steps, such as sequencing and may be performed in addition to selective amplification of DNA comprising a rearranged sequence. For example, this kind of amplification can be performed as part of library preparation (e.g., before selective amplification of DNA comprising a rearranged sequence) and/or after preparation of a targeted library (which would be after selective amplification of DNA comprising a rearranged sequence). Amplification can be primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence based replication.

In some embodiments, the present methods perform dsDNA ligations with T-tailed and C-tailed adapters, which result in amplification of at least 50, 60, 70 or 80% of double stranded nucleic acids before linking to adapters. Preferably the present methods increase the amount or number of amplified molecules relative to control methods performed with T-tailed adapters alone by at least 10, 15 or 20%.

In some embodiments, adapters may be added to the DNA or a subsample thereof. Adapters can be ligated to DNA at any point in the methods herein. In some embodiments, adapters are ligated to the DNA of a sample or subsample thereof prior to annealing primers to the DNA for capture probe generation. In some such embodiments, the adapter-ligated DNA is amplified prior to annealing primers to the DNA for capture probe generation. In some embodiments, adapters are ligated to the DNA of a sample or subsample thereof before the DNA is contacted with the capture probes. In some embodiments, the DNA to which the adapters are ligated is in the same sample or subsample as the DNA used as a template to generate capture probes. In some embodiments, the DNA to which the adapters are ligated is in a different sample or subsample, e.g., a second sample or a second subsample of a first sample, than the DNA used as a template to generate capture probes. In some embodiments, the adapters ligated to DNA captured by the capture probes.

In some embodiments, the primers used to generate capture probes are not complementary to adapters, and the resulting capture probes therefore do not comprise adapters. Adapter-ligated DNA can therefore be selectively amplified in the presence of capture probes that do not comprise adapters. Similarly, adapter-ligated DNA can be separated from DNA that does not comprise adapters.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the single-stranded DNA library preparation is performed in a one-step combined phosphorylation/ligation reaction, e.g., as described in Troll et al., BMC Genomics, 20:1023 (2019), available at https://doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can be delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample or portion of the DNA is partitioned, comprising contacting the DNA with an agent that preferentially binds to nucleic acids bearing an epigenetic modification. The nucleic acids are partitioned into at least two partitioned subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

### Molecular Tagging

In some embodiments, the nucleic acid molecules of the sample may be tagged with sample indexes and/or molecular barcodes (referred to generally as “tags”).

Tags or indexes can be molecules, such as nucleic acids, containing information that indicates a feature of the molecule with which the tag is associated. For example, molecules can bear a sample tag or sample index (which distinguishes molecules in one sample from those in a different sample), a partition tag (which distinguishes molecules in one partition from those in a different partition) and/or a molecular tag/molecular barcode/barcode (which distinguishes different molecules from one another (in both unique and non-unique tagging scenarios).

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments, a tag can comprise one or a combination of barcodes. As used herein, the term “barcode” refers to a nucleic acid molecule having a particular nucleotide sequence, or to the nucleotide sequence itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally, or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. Tags can be used to label the individual polynucleotide population partitions so as to correlate the tag (or tags) with a specific partition. Alternatively, tags can be used in embodiments of the disclosure that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g., by blunt-end ligation or sticky-end ligation), or overlap extension polymerase chain reaction (PCR), among other methods. Such adapters are ultimately joined to the target nucleic acid molecule. In other embodiments, one or more rounds of amplification cycles (e.g., PCR amplification) may be applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after the conversion procedure. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample nucleic acid molecule. In some embodiments, tags are predetermined or random or semi-random sequence oligonucleotides. In some embodiments, the tag(s) may together be less than about 500, 200, 100, 50, 20, 10, 9, 8, 7, 6, 5, 4, 3, 2, or 1 nucleotides in length. Typically, tags are about 5 to 20 or 6 to 15 nucleotides in length. The tags may be linked to sample nucleic acids randomly or non-randomly.

In some embodiments, each sample or partition (discussed below) is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original nucleic acid molecule in the sample, start and stop genomic positions corresponding to the sequence of the original nucleic acid molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original nucleic acid molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit). In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, the assignment of unique or non-unique molecular barcodes in reactions is performed using methods and systems described in, for example, U.S. patents application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, the method includes adding one or more internal control DNAs and forward and reverse primers for amplifying the internal control DNAs. The internal control DNAs may be added before amplification using the primers that anneal upstream and downstream of the rearrangement breakpoints. The forward and reverse primers for amplifying the internal control DNAs may be included with, or added at the same time as, the primers that anneal upstream and downstream of the rearrangement breakpoints. The internal control DNAs may comprise or consist of sequences that do not occur in the genome of the subject, or that do not occur in the genome of the species of which the subject is a member (e.g., the human genome). The forward and/or reverse primers for amplifying the internal control DNAs may comprise sequences that are not complementary to any sequence in the genome of the subject, e.g., the human genome. The internal control DNAs may be used to ensure that the amplification process proceeded as designed. As such, the method may comprise detecting (e.g., sequencing) molecules amplified from and/or captured by the one or more internal control DNAs. The method can comprise comparing an amount of internal control DNAs (e.g., number of molecules or reads detected that correspond to an internal control DNA sequence) to a predetermined threshold, and either rejecting sequencing results if the predetermined threshold is not met or accepting sequencing results if the predetermined threshold is met. The predetermined threshold may be established, e.g., based on historical data or by testing the method on samples of DNA from test subjects, such as healthy volunteers. For example, amplification and detection of the one or more internal control DNAs provides confirmation that the amplification process proceeded properly, thus reducing the likelihood of a false negative.

Analyzing and/or Partitioning DNA

In some instances, a heterogeneous nucleic acid sample is partitioned into two or more partitions (sub-samples). In some embodiments, each partition is differentially tagged. Tagged partitions can then be pooled together for collective sample prep and/or sequencing. The partitioning-tagging-pooling steps can occur more than once, with each round of partitioning occurring based on a different characteristics, and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some embodiments, different procedures are applied to different partitions to determine different characteristics of the initial sample. The nucleic acid, e.g., DNA of at least one partition is subjected to a conversion procedure according to the methods of the disclosure described herein. In some embodiments at least one partition is not subjected to the conversion procedure. Corresponding sequences from the converted and non-converted partitions can be compared to identify single nucleotides that have undergone conversion and therefore identify corresponding modified nucleosides in the initial sample.

For methods that involve a partitioning step, a partition tag (which distinguishes molecules in one partition from those in a different partition) may be included in the adapters or may be added to the sample molecules.

In some embodiments, two or more partitions, e.g., each partition, is/are differentially tagged. Tags can be used to label the individual polynucleotide population partitions so as to correlate the tag (or tags) with a specific partition. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to a molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in genomic region while lower coverage can correlate with lower nucleosome occupancy or a nucleosome depleted region (NDR).

Disclosed methods herein comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Alternative partitioning agents include methyl binding domain (MBDs) and methyl binding proteins (MBPs) as described herein, including proteins such as MeCP2.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation).

In some embodiments, methylation levels can be determined using partitioning, modification-sensitive conversion such as DM-seq, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with capture probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

In some embodiments, methylation is detected using a conversion procedure. Conversion procedures include any technique that differentially alters a first nucleobase but not a second nucleobase in a modification-dependent manner, e.g., being methylated (or hydroxymethylated, or formylated, or carboxylated, etc.) versus unmodified, and/or being modified in one way versus another way (e.g., methylated versus hydroxymethylated). Examples of such conversion procedures include conversion with a deaminase, which converts cytosine to uracil whereas protected modified cytosines (e.g., 5ghmC, 5caC) are not converted. It can be beneficial to include adapters comprising deamination-resistant modified cytosines in DNA that will be subject to deamination, so that the adapters are not affected by deamination. For an exemplary description of such adapters and deamination-resistant modified cytosines, see WO2023/288222A1.

In some embodiments, methylation detection comprises using a methylation-sensitive restriction enzyme (MSRE). For example, a sample, subsample, or portion of a sample can be subjected to digestion with one or more MSREs to cleave unmethylated sequences. Exemplary MSREs include AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, Nael, NotI, NruI, NsbI, PmaCI, Psp1406I, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I. In some embodiments, the portion of the sample that is contacted with one or more MSREs comprises hypermethylated DNA, or is or comprises a hypermethylated DNA partition, which may be obtained as described elsewhere herein.

In some embodiments, DNA fragmentation is detected by determining the endpoints and/or midpoints of sequenced fragments of DNA (e.g., cfDNA). For example, differences in fragmentation patterns may occur depending on whether the fragments originated from a tumor or from healthy cells. To detect tumor-cell derived DNA of cfDNA based on fragmentation, the presence or absence of an increased level of abnormal fragments can be determined at regions with copy-number amplifications, (e.g., proportional to the degree of amplification), e.g., where the increase and abnormality are relative to control or healthy samples.

In some embodiments, a sample or subsample (e.g., a first, second, or third subsample prepared by partitioning a sample as described herein, such as on the basis of a level of a cytosine modification, such as methylation, e.g., 5-methylation, such as of cytosine) is contacted with a methylation-dependent nuclease or methylation-sensitive nuclease. Unless otherwise indicated, where partitioning is performed on the basis of a cytosine modification, the first subsample is the subsample with a higher level of the modification; the second subsample is the subsample with a lower level of the modification; and, when present, the third subsample has a level of the modification intermediate between the first and second subsamples.

As discussed above, partitioning procedures may result in imperfect sorting of DNA molecules among the subsamples. The choice of a methylation-dependent nuclease or methylation-sensitive nuclease can be made so as to degrade nonspecifically partitioned DNA. For example, the second subsample can be contacted with a methylation-dependent nuclease, such as a methylation-dependent restriction enzyme. This can degrade nonspecifically partitioned DNA in the second subsample (e.g., methylated DNA) to produce a treated second subsample. Alternatively or in addition, the first subsample can be contacted with a methylation-sensitive endonuclease, such as a methylation-sensitive restriction enzyme, thereby degrading nonspecifically partitioned DNA in the first subsample to produce a treated first subsample. Degradation of nonspecifically partitioned DNA in either or both of the first or second subsamples is proposed as an improvement to the performance of methods that rely on accurate partitioning of DNA on the basis of a cytosine modification, e.g., to detect the presence of aberrantly modified DNA in a sample, to determine the tissue of origin of DNA, and/or to determine whether a subject has cancer. For example, such degradation may provide improved sensitivity and/or simplify downstream analyses. In general, where nonspecifically partitioned DNA would be hypermethylated, such as in a hypomethylated partition, a methylation-dependent nuclease, such as a methylation-dependent restriction enzyme, should be used. Conversely, where nonspecifically partitioned DNA would be hypomethylated, such as in a hypermethylated partition, a methylation-sensitive nuclease, such as a methylation-sensitive restriction enzyme, should be used. Methylation-dependent nucleases, such as methylation-dependent restriction enzymes, preferentially cut methylated DNA relative to unmethylated DNA, while methylation-sensitive nucleases, such as methylation-sensitive restriction enzymes, preferentially cut unmethylated DNA relative to methylated DNA.

In contacting a subsample with a nuclease, one or more nucleases can be used. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting the second subsample with more than one methylation-dependent restriction enzyme can more completely degrade nonspecifically partitioned hypermethylated DNA. Similarly, contacting the first subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used. In some embodiments, the methylation-dependent nuclease comprises FspEI. In some embodiments, the methylation-dependent nuclease comprises FspEI and MspJI, e.g., used sequentially.

In some embodiments, a methylation-sensitive nuclease comprises one or more of AatII, AccII, AciI, Aor13HI, Aor15HI, BspT104I, BssHII, BstUI, Cfr10I, ClaI, CpoI, Eco52I, HaeII, HapII, HhaI, Hin6I, HpaII, HpyCH4IV, MluI, MspI, NaeI, NotI, NruI, NsbI, PmaCI, Psp1406I, PvuI, SacII, SalI, SmaI, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, HpaII and Hin6I.

In some embodiments, FspEI is used for digesting the nucleic acid molecules in at least one subsample (e.g., a hypomethylated partition). In some embodiments, BstUI, HpaII and Hin6I are used for digesting the nucleic acid molecules in at least one subsample (e.g., a hypermethylated partition) and FspEI is used for digesting the nucleic acid molecules in at least one other subsample (e.g., a hypomethylated partition). In embodiments involving an intermediately methylated partition, the nucleic acid molecules therein may be digested with a methylation-sensitive nuclease or a methylation-dependent nuclease. In some embodiments, the nucleic acid molecules in an intermediately methylated partition are digested with the same nuclease(s) as the hypermethylated partition. For example, the intermediately methylated partition may be pooled with the hypermethylated partition and then the pooled partitions may be subjected to digestion. In some embodiments, the nucleic acid molecules in an intermediately methylated partition are digested with the same nuclease(s) as the hypomethylated partition. For example, the intermediately methylated partition may be pooled with the hypomethylated partition and then the pooled partitions may be subjected to digestion.

In some embodiments, a subsample is contacted with a nuclease as described above after a step of tagging or attaching adapters to both ends of the DNA. The tags or adapters can be resistant to cleavage by the nuclease using any of the approaches described above. In this approach, cleavage can prevent the nonspecifically partitioned molecule from being carried through the analysis because the cleavage products lack tags or adapters at both ends.

Alternatively, a step of tagging or attaching adapters can be performed after cleavage with a nuclease as described above. Cleaved molecules can be then identified in sequence reads based on having an end (point of attachment to tag or adapter) corresponding to a nuclease recognition site. Processing the molecules in this way can also allow the acquisition of information from the cleaved molecule, e.g., observation of somatic mutations. When tagging or attaching adapters after contacting the subsample with a nuclease, and low molecular weight DNA such as cfDNA is being analyzed, it may be desirable to remove high molecular weight DNA (such as contaminating genomic DNA) from the sample before the contacting step. It may also be desirable to use nucleases that can be heat-inactivated at a relatively low temperature (e.g., 65° C. or less, or 60° C. or less) to avoid denaturing DNA, in that denaturation may interfere with subsequent ligation steps.

Where a sample is partitioned into three subsamples, including a third subsample containing intermediately methylated molecules, the third subsample is in some embodiments contacted with a methylation-sensitive nuclease. Such a step may have any of the features described elsewhere herein with respect to contacting steps, and may be performed before or after a step of tagging or attaching adapters as discussed above. In some embodiments, the first and third subsamples are combined before being contacted with a methylation-sensitive nuclease. Such a step may have any of the features described elsewhere herein with respect to contacting steps, and may be performed before or after a step of tagging or attaching adapters as discussed above. In some embodiments, the first and third subsamples are differentially tagged before being combined.

Alternatively, where a sample is partitioned into three subsamples, including a third subsample containing intermediately methylated molecules, the third subsample is in some embodiments contacted with a methylation-dependent nuclease. Such a step may have any of the features described elsewhere herein with respect to contacting steps, and may be performed before or after a step of tagging or attaching adapters as discussed above. In some embodiments, the second and third subsamples are combined before being contacted with a methylation-dependent nuclease. Such a step may have any of the features described elsewhere herein with respect to contacting steps, and may be performed before or after a step of tagging or attaching adapters as discussed above. In some embodiments, the second and third subsamples are differentially tagged before being combined.

In some embodiments, the DNA is purified after being contacted with the nuclease, e.g., using SPRI beads. Such purification may occur after heat inactivation of the nuclease. Alternatively, purification can be omitted; thus, for example, a subsequent step such as amplification can be performed on the subsample containing heat-inactivated nuclease. In another embodiment, the contacting step can occur in the presence of a purification reagent such as SPRI beads, e.g., to minimize losses associated with tube transfers. After cleavage and heat inactivation, the SPRI beads can be re-used for cleanup by adding molecular crowding reagents (e.g., PEG) and salt.

In some embodiments, where a conversion procedure is performed on a sample or subsample, the subsequent capturing of one or more target region sets (e.g., at least an epigenetic target region set) from that sample or subsample uses capture probes that comprise probes specific for a modification state (e.g., of at least one base in the sequence to which the probe hybridizes), e.g., complementary to target sequences that have undergone conversion (e.g., conversion of modified or unmodified cytosines to uracils or analogs thereof, such as DHU, that preferentially pair with adenine) or that have not undergone conversion, as desired. As such, the probes can be specific for sequences in which a modification of interest, such as methylation, was or was not present. In some embodiments, where a modification sensitive conversion is performed on a sample or subsample, the subsequent capturing of one or more target region sets (e.g., at least an epigenetic target region set) from that sample or subsample uses capture probes that comprise probes that can hybridize to target sequences regardless of modification state (e.g., comprise a promiscuously pairing nucleobase at a position that may or may not have undergone conversion of modified or unmodified cytosines to uracils or analogs thereof, such as DHU, that preferentially pair with adenine; for example, inosine can pair with C or U).

In some embodiments, the methods comprise preparing a pool comprising at least a portion of the DNA of the second subsample (also referred to as the hypomethylated partition) and at least a portion of the DNA of the first subsample (also referred to as the hypermethylated partition). Target regions, e.g., including epigenetic target regions and/or sequence-variable target regions, may be captured from the pool. The steps of capturing a target region set from at least a portion of a subsample described elsewhere herein encompass capture steps performed on a pool comprising DNA from the first and second subsamples. A step of amplifying DNA in the pool may be performed before capturing target regions from the pool. The capturing step may have any of the features described elsewhere herein.

The epigenetic target regions may show differences in methylation levels and/or fragmentation patterns depending on whether they originated from a tumor or from healthy cells, or what type of tissue they originated from, as discussed elsewhere herein. The sequence-variable target regions may show differences in sequence depending on whether they originated from a tumor or from healthy cells.

Analysis of epigenetic target regions from the hypomethylated partition may be less informative in some applications than analysis of sequence-variable target-regions from the hypermethylated and hypomethylated partitions and epigenetic target regions from the hypermethylated partition. As such, in methods where sequence-variable target-regions and epigenetic target regions are being captured, the latter may be captured to a lesser extent than one or more of the sequence-variable target-regions from the hypermethylated and hypomethylated partitions and epigenetic target regions from the hypermethylated partition. For example, sequence-variable target regions can be captured from the portion of the hypomethylated partition not pooled with the hypermethylated partition, and the pool can be prepared with some (e.g., a majority, substantially all, or all) of the DNA from the hypermethylated partition and none or some (e.g., a minority) of the DNA from the hypomethylated partition. Such approaches can reduce or eliminate sequencing of epigenetic target regions from the hypomethylated partition, thereby reducing the amount of sequencing data that suffices for further analysis.

In some embodiments, including a minority of the DNA of the hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of the hypomethylated partition, e.g., less than about 50% of the DNA of the hypomethylated partition, such as less than or equal to about 45%, 40%, 35%, 30%, 25%, 20%, 15%, 10%, or 5% of the DNA of the hypomethylated partition. In some embodiments, the pool comprises about 5%-25% of the DNA of the hypomethylated partition. In some embodiments, the pool comprises about 10%-20% of the DNA of the hypomethylated partition. In some embodiments, the pool comprises about 10% of the DNA of the hypomethylated partition. In some embodiments, the pool comprises about 15% of the DNA of the hypomethylated partition. In some embodiments, the pool comprises about 20% of the DNA of the hypomethylated partition.

In some embodiments, the pool comprises a portion of the hypermethylated partition, which may be at least about 50% of the DNA of the hypermethylated partition. For example, the pool may comprise at least about 55%, 60%, 65%, 70%, 75%, 80%, 85%, 90%, or 95% of the DNA of the hypermethylated partition. In some embodiments, the pool comprises 50-55%, 55-60%, 60-65%, 65-70%, 70-75%, 75-80%, 80-85%, 85-90%, 90-95%, or 95-100% of the DNA of the hypermethylated partition. In some embodiments, the second pool comprises all or substantially all of the hypermethylated partition.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise capturing at least a first set of target regions from the first pool, e.g., wherein the first pool is as set forth in any of the embodiments above. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this capture step. In some embodiments, capturing the first set of target regions from the first pool comprises contacting the DNA of the first pool with a first set of capture probes. In some embodiments, the first set of capture probes comprises target-binding probes specific for the sequence-variable target regions. In some embodiments, the first set of capture probes comprises target-binding probes specific for the sequence-variable target regions, hypomethylation variable target regions and/or fragmentation variable target regions.

In some embodiments, the methods comprise capturing a second set of target regions or plurality of sets of target regions from the second pool, e.g., wherein the first pool is as set forth in any of the embodiments above. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this capture step. In some embodiments, capturing the second plurality of sets of target regions from the second pool comprises contacting the DNA of the first pool with a second set of capture probes, wherein the second set of capture probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is captured from the second pool but not from the first pool. In some embodiments, a plurality of hypermethylation variable target regions are captured from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

In some embodiments, the partitions of DNA are desalted and concentrated in preparation for enzymatic steps of library preparation. Sequences that comprise structural variations may tend to be hypomethylated. Accordingly, in some embodiments, the DNA contacted with a plurality of primers comprising at least two primers that anneal in an anti-parallel orientation to a rearranged sequence of DNA is from or comprises at least a portion of a hypomethylated partition. The DNA from or comprising at least a portion of hypomethylated partition may or may not be combined with DNA from or comprising at least a portion of one or more other partitions, such as an intermediate partition or a hypermethylated partition.

### Amplification

Sample nucleic acids flanked by adapters can be amplified by PCR and other amplification methods. Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence based replication.

In some embodiments, the present methods perform dsDNA ligations with T-tailed and C-tailed adapters, which result in amplification of at least 50, 60, 70 or 80% of double stranded nucleic acids. Preferably the present methods increase the amount or number of amplified molecules relative to control methods performed with T-tailed adapters alone by at least 10, 15 or 20%.

In some embodiments, sample nucleic acids are amplified before sequencing. Amplification may be before and/or after the conversion step. Amplification may in some cases be before one or more capture steps. In some embodiments, the ligating occurs before or simultaneously with amplification.

In some embodiments, amplification is primed by primer binding to primer binding site(s) in the adapter oligonucleotide(s). The known modified nucleosides of the adapter(s) may be outside of the primer binding site(s), i.e., the known modified nucleosides used for the quality control method are not in the primer-binding sites or bound by the amplification primers.

### Enriching, Capturing and Using Capture Probes; Target Regions

Nucleic acids in a sample can be subject to a capture step, in which molecules having target sequences are captured for subsequent analysis. Capture may be performed using any suitable approach known in the art. Target capture can involve use of a bait set comprising oligonucleotide baits labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, DNA is captured that comprises a region comprising a type-specific epigenetic variation. In some embodiments, the variations are present in healthy cells but not normally present in the sample type, such as a blood sample. In some embodiments, the variations are present in aberrant cells (e.g., hyperplastic, metaplastic, or neoplastic cells).

In some embodiments, a first captured epigenetic target region set captured from a sample or first subsample comprises hypermethylation variable target regions. In some embodiments, the hypermethylation variable target regions show type-specific hypermethylation in healthy cfDNA from one or more related cell or tissue types. Without wishing to be bound by any particular theory, the presence of cancer cells may increase the shedding of DNA into the bloodstream (e.g., from the cancer and/or the surrounding tissue). As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, the methods herein comprise capturing a second captured epigenetic target region set from a sample or second subsample. In some embodiments, the second epigenetic target region set comprises hypomethylation variable target regions. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

Additionally, captured target region sets may comprise DNA corresponding to a sequence-variable target region set. The captured sets may be combined to provide a combined captured set.

In some embodiments in which a captured set comprising DNA corresponding to a sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11-to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

In some embodiments, the DNA that is captured comprises intronic regions. In some embodiments, the intronic regions comprise one or more introns likely to differentiate DNA from neoplastic (e.g., tumor or cancer) cells and from healthy cells, e.g., non-neoplastic circulating cells. For example, an intron comprising a rearrangement known to be present in some neoplastic cells and absent from healthy cells can be used to differentiate DNA from neoplastic (e.g., tumor or cancer) cells and from healthy cells. In some embodiments, the rearrangement is a translocation.

In some embodiments, captured intronic regions have a footprint of at least 30 bp, e.g., at least 100 bp, at least 200 bp, at least 500 bp, at least 1 kb, at least 2 kb, at least 5 kb, at least 10 kb, at least 20 kb, at least 50 kb, at least 200 kb, at least 300 kb, or at least 400 kb. In some embodiments, the intronic target region set has a footprint in the range of 30 bp-1000 kb, e.g., 30 bp-100 bp, 100 bp-200 bp, 200 bp-500 bp, 500 bp-1 kb, 1 kb-2 kb, 2 kb-5 kb, 5 kb-10 kb, 10 kb-20 kb, 20 kb-50 kb, 50 kb-100 kb, 100-200 kb, 200-300 kb, 300-400 kb, 400-500 kb, 500-600 kb, 600-700 kb, 700-800 kb, 800-900 kb, and 900-1,000 kb.

Exemplary rearrangements, such as intronic translocations that can be detected using the methods described herein include but are not limited to translocations wherein at least one of the two genes involved in the translocation is a receptor tyrosine kinase. Exemplary translocation products are the BCR-ABL fusion, and fusions comprising any of ALK, FGFR2, FGFR3, NTRK1, RET, or ROS1.

In some embodiments, the DNA that is captured comprises target regions having a type-specific epigenetic variation. In some embodiments, an epigenetic target region set consists of target regions having a type-specific epigenetic variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell or tissue types but not to the extent observed in the one or more related cell or tissue types. In some embodiments, the hypermethylation variable target regions show lower methylation in healthy cfDNA than in at least one other tissue type. In some embodiments, the hypermethylation variable target regions show even higher methylation in cfDNA from a diseased cell of the one or more related cell or tissue types. In some embodiments, target regions comprise hypermethylated regions with aberrantly high copy number. In some such embodiments, the target regions are hypermethylated in healthy and diseased colon tissue and have aberrantly high copy number in pre-cancerous or cancerous colon tissue. Examples of such target regions are shown in Table 1 below.

TABLE 1
Hypermethylated target regions with aberrantly
high copy number in colon cancer or pre-cancer

| Chromosomal region | |
|---|---|
| with copy number gain | Genes comprising DMRs within the chromosomal region |
| 7p21.3-15.1 | VWDE, TWIST1, DNAH1, GPNMB, NPY, GSDME, NFE2L3, |
| | HOXA1, HOXA7, EVX1, CREB5 |
| 8q13.3-24.3 | PRDM14, MSC, TRPA1, SBSPON, CRISPLD1, ZFHX4, RALYL, |
| | MMP16, C8orf88, RUNX1T1, CDH17, GDF6, SDC2, OSR2, |
| | PABPC1, ZNF706, RIMS2, ZFPM2, SYBU, CSMD3, TRPS1, |
| | AARD, TNFRSF11B, MAL2, COL14A1, POU5F1B, ADCY8, |
| | ST3GAL1, TRAPPC9, AGO2, ADGRB1, LY6E, LY6H, GLI4, |
| | MAFA, TSTA3, PLEC, OPLAH, FOXH1 |
| 13q14.1-33.3 | ELF1, RGCC, TNFSF11, SERP2, RB1, CNMD, PCDH17, EDNRB, |
| | POU4F1, SLITRK5, GPC6, SOX21, CLDN10, STK24, ZIC2, |
| | NALCN, FGF14, MYO16, COL4A1, COL4A2, SOX1, F7 |
| 20q12-13.33 | MAFB, LPIN3, EMILIN3, PTPRT, TOX2, GDAP1L1, HNF4A, |
| | MMP9, CDH22, PREX1, ZNFX1, PARD6B, ZFP64, DOK5, |
| | FAM210B, TFAP2C, RBM38, CTCFL, GNAS, PHACTR3, CDH4, |
| | GATA5, BHLHE23, YTHDF1, NKAIN4, CHRNA4, KCNQ2, |
| | GMEB2 |

TABLE 2
Exemplary Hypermethylation Target Regions
based on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

In some embodiments, a captured epigenetic target region set captured from a sample or subsample comprises hypomethylation variable target regions. In some embodiments, the hypomethylation variable target regions are exclusively hypomethylated in one or more related cell or tissue types. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell or tissue types but not to the extent observed in the one or more cell or tissue types. In some embodiments, the hypomethylation variable target regions show higher methylation in healthy cfDNA than in at least one other tissue type.

Without wishing to be bound by any particular theory, in an individual with cancer, proliferating or activated immune cells and/or dying cancer cells may shed more DNA into the bloodstream than immune cells in a healthy individual and/or healthy cells of the same tissue type, respectively. As such, the distribution of cell type and/or tissue of origin of cfDNA may change upon carcinogenesis. Thus, the presence and/or levels of cfDNA originating from certain cell or tissue types can be an indicator of disease. Variations in hypermethylation and/or hypomethylation can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set. The sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above and in WO 2020/160414.

In some embodiments, a collection of capture probes is used in methods described herein, e.g., comprising capture probes prepared by any method disclosed herein for doing so. In some embodiments, the collection of capture probes further comprises target-binding probes specific for a sequence-variable target region set and/or target-binding probes specific for an epigenetic target region set. In some embodiments, the capture yield of the target-binding probes specific for the sequence-variable target region set is higher (e.g., at least 2-fold higher) than the capture yield of the target-binding probes specific for the epigenetic target region set. In some embodiments, the collection of capture probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the capture yield of the target-binding probes specific for the epigenetic target region set.

In some embodiments, the collection of capture probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of capture probes is configured to have a capture yield specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the capture probes specific for the sequence-variable target region set are present at a higher concentration than the capture probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the capture probes specific for the sequence-variable target region set have a higher affinity for their targets than the capture probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39:8740-8751 (2011); Freier et al., Nucleic Acids Res. 25:4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the capture probes specific for the sequence-variable target region set have modifications that increase their affinity for their targets. In some embodiments, alternatively or additionally, the capture probes specific for the epigenetic target region set have modifications that decrease their affinity for their targets. In some embodiments, the capture probes specific for the sequence-variable target region set have longer average lengths and/or higher average melting temperatures than the capture probes specific for the epigenetic target region set. These embodiments may be combined with each other and/or with differences in concentration as discussed above to achieve a desired fold difference in capture yield, such as any fold difference or range thereof described above.

In some embodiments, the capture probes comprise a capture moiety. The capture moiety may be any of the capture moieties described herein, e.g., biotin. In some embodiments, the capture probes are linked to a solid support, e.g., covalently or non-covalently such as through the interaction of a binding pair of capture moieties. In some embodiments, the solid support is a bead, such as a magnetic bead.

In some embodiments, the capture probes specific for the sequence-variable target region set and/or the capture probes specific for the epigenetic target region set are a capture probe set as discussed above, e.g., probes comprising capture moieties and sequences selected to tile across a panel of regions, such as genes.

In some embodiments, the capture probes are provided in a single composition. The single composition may be a solution (liquid or frozen). Alternatively, it may be a lyophilizate.

Alternatively, the capture probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

### Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

### CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

### Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

### Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

### Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 3. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 3. In some embodiments, probes specific for the sequence-variable target region set comprise probes specific for at least 1, at least 2, at least 3, at least 4, at least 5, or 6 of the fusions of Table 3. In some embodiments, probes specific for the sequence-variable target region set comprise probes specific for at least a portion of at least 1, at least 2, or 3 of the indels of Table 3. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 4. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 4. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 5.

TABLE 3

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 5

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS / Indels |
| GATA3 | chr10 | 8115692 | 8116002 | 310 | 6 | SS / Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5: 1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175 / Deletions |
| TP53 | chr17 | 7579301 | 7579600 | 299 | 4 | |
| | | | | 12574 | | |
| | | | | (total target | | |
| | | | | region) | | |
| | | | | 16330 | | |
| | | | | (total probe | | |
| | | | | coverage) | | |

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Sequencing

In some embodiments, the method comprises sequencing at least a portion of the treated DNA. In general, sample nucleic acids flanked by adapters with or without prior amplification can be subject to sequencing. Sequencing methods include, for example, Sanger sequencing, high-throughput sequencing, pyrosequencing, sequencing-by-synthesis, long-read sequencing (also known as single-molecule sequencing or third generation sequencing), nanopore sequencing (a type of long-read sequencing), 5-letter sequencing or 6-letter sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing unit can also include multiple sample chambers to enable processing of multiple runs simultaneously.

In some embodiments, sequencing comprises detecting and/or distinguishing unmodified and modified nucleobases. For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing can facilitate direct detection of, e.g., 5-methylcytosine and 5-hydroxymethylcytosine as well as unmodified cytosine (Weirather J L, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adaptors to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.) SMRT sequencing can thus be used to detect base modifications such as 5-caC, 4mC, 5mC, 5hmC, 6 mA, and 8oxoG (Gouil & Keniry Essays in Biochemistry (2019) 63 639-648). Accordingly, in some embodiments, the sequencing comprises SMRT sequencing. In such embodiments, the end repair may be performed using dNTPs, which comprise 5-caC, 4mC, 5mC, 5hmC, 6 mA, and/or 8oxoG.

Some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore.

One example of a nanopore-based single molecule sequencing system is that commercialized by Oxford Nanopore Technologies (ONT). (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adaptor. Therefore, the DNA template passes through the nanopore, followed by a hairpin and finally the complement. The raw read can be split into two “ID” reads (“template” and “complement”) by removing the adaptor. The consensus sequence of two “ID” reads is a “2D” read with a higher accuracy.

Nanopore sequencing can be used to detect base modifications including 5-caC, 5mC, 5hmC, 6 mA, BrdU, FldU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the sequencing comprises nanopore sequencing. In such embodiments, the end repair may be performed using dNTPs, which comprise 5-caC, 4mC, 5mC, 5hmC, 6 mA, BrdU, FldU, IdU, and/or EdU.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adaptors at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil- and/or dihydrouracil-tolerant amplification methods, such as PCR using a uracil- and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of, for example, at least 5%, 10%, 15%, 20%, 25%, 30%, 40%, 50%, 60%, 70%, or 80% of the genome. Sequence coverage can performed on, for example, at least 5, 10, 20, 70, 100, 200 or 500 different genes, or up to, for example, 5000, 2500, 1000, 500 or 100 different genes.

Simultaneous sequencing reactions may be performed using multiplex sequencing. In some embodiments, cell-free nucleic acids may be sequenced with at least, for example, 1000, 2000, 3000, 4000, 5000, 6000, 7000, 8000, 9000, 10000, 50000, or 100,000 sequencing reactions. In other embodiments, cell-free nucleic acids may be sequenced with less than, for example, 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least, for example, 1000, 2000, 3000, 4000, 5000, 6000, 7000, 8000, 9000, 10000, 50000, or 100,000 sequencing reactions. In other cases, data analysis may be performed on less than, for example, 1000, 2000, 3000, 4000, 5000, 6000, 7000, 8000, 9000, 10000, 50000, or 100,000 sequencing reactions. An exemplary read depth is 1000-50000 or 1000-10000 or 1000-20000 reads per locus (base).

In general, sequencing of epigenetic target regions, e.g., to analyze a modified nucleoside profile of DNA, requires a lesser depth of sequencing than sequencing of a sequence-variable target region, e.g. for analysis of mutations. Hence, lesser sequencing depths, as described herein, may in some cases be adequate for the methods described herein.

### Differential Depth of Sequencing

In some embodiments, nucleic acids corresponding to the sequence-variable target region set are sequenced to a greater depth of sequencing than nucleic acids corresponding to the epigenetic target region set. For example, the depth of sequencing for nucleic acids corresponding to the sequence-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

### Amplification of DNA Comprising Adapters

Sample nucleic acids flanked by adapters can be amplified by PCR and other amplification methods. Such amplification can be used to increase the amount of DNA available for subsequent steps, such as sequencing. For example, this kind of amplification can be performed as part of library preparation and/or after preparation of a targeted library. The amplification may be performed after a conversion procedure. Amplification can be primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters, which result in amplification of at least 50, 60, 70 or 80% of double stranded nucleic acids before linking to adapters. Preferably the present methods increase the amount or number of amplified molecules relative to control methods performed with T-tailed adapters alone by at least 10, 15 or 20%.

### Exemplary Applications

The methods presented herein may be used as part of any method that benefits from obtaining a modified nucleoside profile of DNA in any sample.

One exemplary application of the methods of the disclosure is using the modified nucleoside profile in diagnosing and prognosing cancer or other genetic diseases or conditions.

Hence, in some embodiments, a method described herein comprises identifying or predicting the presence or absence of DNA produced by a tumor (or neoplastic cells, or cancer cells), determining the probability that a test subject has a tumor or cancer, and/or characterizing a tumor, neoplastic cells or cancer as described herein.

### 1. Cancer and Other Diseases; Cell Type Quantification

The present methods can be used to diagnose presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may increase the amount of rare mutations detected in subject's blood if the treatment is successful as more cancers may die and shed DNA. In other examples, this may not occur. In another example, certain treatment options may be correlated with genetic profiles of cancers over time. This correlation may be useful in selecting a therapy. In some embodiments, target regions (e.g., hypermethylation variable epigenetic target regions) are analyzed to determine whether they show methylation (e.g., hypermethylation) characteristics of tumor cells or cells that do not ordinarily contribute significantly to cfDNA and/or target regions (e.g., hypomethylation variable target regions) are analyzed to determine whether they show methylation (e.g., hypomethylation) characteristic of tumor cells or cells that do not ordinarily contribute significantly to cfDNA. In some embodiments, successful treatment options may result in changes in levels of different immune cell types (including rare immune cell types), and/or increases in the amount of target proteins, copy number variation, rare mutations, and/or cancer-related epigenetic signatures (such as hypermethylated regions or hypomethylated regions) detected in, e.g., a sample from a subject, such as detected in a subject's blood (such as in DNA isolated from a buffy coat sample or any other sample comprising cells, such as in a blood sample (e.g., a whole blood sample, a plasma sample, a leukapheresis sample, or a PBMC sample) from the subject) if the treatment is successful as more cancer cells may die and shed DNA, or, e.g., if a successful treatment results in an increase or decrease in the quantity of a specific protein in the blood and an unsuccessful treatment results in no change.

Additionally, if a cancer is observed to be in remission after treatment, the present methods can be used to monitor the likelihood of residual disease or the likelihood of recurrence of disease.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

In some embodiments, the methods and systems disclosed herein may be used to identify customized or targeted therapies to treat a given disease or condition in patients based on the classification of a nucleic acid variant as being of somatic or germline origin.

Typically, the disease under consideration is a type of cancer, such as any referred to herein. The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

In some embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, such as 5mC and/or 5hmC profiles. Hence, the present methods can in some cases be used in combination with methods used to detect other genetic/epigenetic variations, e.g. in a method of detecting or characterizing a cancer or other methods described herein.

In some embodiments, a method described herein comprises identifying the presence of target regions and/or DNA produced by a tumor (or neoplastic cells, or cancer cells) or by precancer cells. In some embodiments, a method described herein comprises determining the level of target regions and/or identifying the presence of DNA produced by a tumor (or neoplastic cells, or cancer cells) or by precancer cells. In some embodiments, determining the level of target regions comprises determining either an increased level or decreased level of target regions, wherein the increased or decreased level of target regions is determined by comparing the level of target regions with a threshold level/value.

Genetic and/or epigenetic data can also be used for characterizing a specific form of cancer. Cancers are often heterogeneous in both composition and staging. Genetic and/or epigenetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic and/or epigenetic profile of extracellular polynucleotides, such as cfDNA, derived from the subject, wherein the genetic and/or epigenetic profile comprises a plurality of data resulting from copy number variation and rare mutation analyses. In some embodiments, an abnormal condition is cancer, e.g. as described herein. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer or precancer, or has a metastasis, that is related to changes in proportions of types of immune cells.

The present methods can be used to generate or profile, fingerprint or set of data that is a summation of genetic and/or epigenetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein comprises detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells. Comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of Determining a Risk of Cancer Recurrence in a Test Subject and/or Classifying a Subject as being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting a sample (such as DNA, such as DNA originating or derived from a tumor cell) from the subject diagnosed with the cancer at one or more preselected timepoints following one or more previous cancer treatments to the subject. The subject may be any of the subjects described herein. The sample may comprise chromatin, cfDNA, or other cell materials. The sample, such as the DNA sample, may be a tissue sample. The DNA may be DNA, such as cfDNA, from a blood sample (e.g., a whole blood sample, a buffy coat sample, a leukapheresis sample, or a PBMC sample). The DNA may comprise DNA obtained from a tissue sample.

Any of such methods may comprise contacting the sample or a subsample thereof with a plurality of primers, generating capture probes, capturing and detecting the presence or level of at least one structural variation according to any of the embodiments as described herein. In some embodiments, the methods may comprise contacting the sample or a subsample thereof with a plurality of capture probes specific for members of an epigenetic target region set according to any of the embodiments as described herein. In some embodiments, the capture probes comprise capture probes generated using a sample obtained from the same subject at an earlier timepoint. Any of such methods may comprise capturing a plurality of sets of target regions from DNA from the subject, wherein the plurality of target region sets comprises a sequence-variable target region set and an epigenetic target region set, whereby a captured set of DNA molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA, such as cfDNA, originating or derived from a tumor cell may be performed according to any of the embodiments thereof described elsewhere herein.

Methods of determining a risk of cancer recurrence in a subject may comprise determining a cancer recurrence score that is indicative of the presence or absence, or amount, of the DNA, such as genomic regions of interest and target regions, originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, sequence-variable target region sequences are obtained, and determining the cancer recurrence score may comprise determining at least a first subscore indicative of the amount of the levels of particular immune cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, epigenetic target region sequences are obtained, and determining the cancer recurrence score comprises determining a second subscore indicative of the amount of molecules (obtained from the epigenetic target region sequences) that represent an epigenetic state different from DNA found in a corresponding sample from a healthy subject (e.g., DNA, such as cfDNA, found in a blood sample (e.g., a whole blood sample, a buffy coat sample, a leukapheresis sample, or a PBMC sample) from a healthy subject, or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer (such as with a metastasis), e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or more of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence-variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence-variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10-7 is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a subscore indicative of the amount of SNVs, insertions/deletions, CNVs and/or fusions present in sequence-variable target region sequences and a subscore indicative of the amount of abnormal molecules in epigenetic target region sequences, and combining the subscores to provide the cancer recurrence score. Where the subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, the set of sequence information comprises sequence-variable target region sequences and epigenetic target region sequences, and determining the cancer recurrence score comprises determining a first subscore indicative of the levels of particular immune cell types, a second subscore indicative of the amount of SNVs, insertions/deletions, CNVs and/or fusions present in sequence-variable target region sequences and a third subscore indicative of the amount of abnormal molecules in epigenetic target region sequences, and combining the first, second, and third subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening). In some embodiments, monitoring comprises analysis of at least two samples collected from a subject at at least two different time points as described herein.

The methods according to the present disclosure can be useful in predicting a subject's response to a particular treatment option, such as over a period of time. As described elsewhere herein, successful treatment options may increase the amount of cancer associated DNA sequences detected in a subject's blood, such as if the treatment is successful as more cancers may die and shed DNA. In such examples, certain treatment options may be correlated with genetic profiles of cancers over time. This correlation may be useful in selecting a therapy. In some embodiments, successful treatment options may result in an increase or decrease in the levels of different immune cell types (including rare immune cell types), and/or an increase or decrease in the levels of a specific protein or proteins and/or a specific DNA sequence (e.g., of a CDR3), such as in the blood, and an unsuccessful treatment may result in no change. In other examples, this may not occur.

As disclosed herein, in some embodiments, quantities of each of a plurality of cell types, such as immune cell types, are determined based on sequencing and analysis (such as determination of epigenetic and/or genomic signatures) of DNA isolated from at least one sample comprising cells (such as a tissue sample or a blood sample, e.g., a whole blood sample, a buffy coat sample, a leukapheresis sample, or a PBMC sample) from a subject. In some embodiments, differences in levels and/or presence of particular genetic and/or epigenetic signatures in DNA isolated from blood samples from a subject can be used to quantify cell types, such as immune cell types, within the sample. Thus, a comparison of the disclosed genetic and/or epigenetic signatures in DNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples (such as a tissue sample or a blood sample, e.g., a whole blood sample, a buffy coat sample, a leukapheresis sample, or a PBMC sample) collected from a subject at one or more timepoints in comparison to a selected baseline value or reference standard (or a selected set of baseline values or reference standards). A baseline value or reference standard may be a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples (such as a tissue sample or a blood sample, e.g., a whole blood sample, a buffy coat sample, a leukapheresis sample, or a PBMC sample) may be collected from a subject at two or more timepoints, to assess changes in cell types (such as changes in quantities of cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one tissue sample and/or at least one blood sample, e.g., a whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples (such as one or more tissue, whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more tissue samples or blood samples, e.g., or one or more buffy coat samples, whole blood samples, leukapheresis samples, or PBMC samples) are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering customized therapies, such as customized therapies to patients. In some embodiments, determination of the levels of particular immune cell types, including rare immune cell types, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In some embodiments, e.g., where genetic variants are detected, therapy is customized based on the status of a nucleic acid variant as being of somatic or germline origin. In some embodiments, determination of the levels of particular cell types, e.g., immune cell types, including rare immune cell types, facilitates selection of appropriate treatment.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a buffy coat sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject. In some embodiments, determination of the metastasis site facilitates selection of appropriate treatment.

Thus, in some embodiments, quantities of each of one or more of a particular genetic and/or epigenetic signature (e.g., quantities of fusions, indels, SNPs, CNVs, and/or rare mutations, and/or cancer-related epigenetic signatures (such as specific (e.g., DMRs) or global hypermethylated or hypomethylated regions, and/or fragmentation variable regions)) in DNA from a subject's blood (such as in DNA (e.g., cfDNA) isolated from a blood sample (e.g., a whole blood sample) from the subject)) are determined based on sequencing and analysis. In some embodiments, quantities of each of a plurality of cell types, such as immune cell types, are determined based on sequencing and analysis (such as determination of epigenetic and/or genomic signatures) of DNA isolated from at least one sample comprising cells (such as blood sample (e.g., a whole blood sample, a buffy coat sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic and/or epigenetic signatures in DNA isolated from blood samples from a subject can be used to quantify cell types, such as immune cell types, within the sample. Thus, a comparison of one or more genetic and/or epigenetic signatures in DNA isolated from blood samples collected from a subject at two or more time points can be used to monitor changes in the one or more signatures and/or the one or more cell type quantities in the subject under different conditions (such as prior to and after a treatment), or over time (e.g., as part of a preventative health monitoring program).

In some embodiments, therapy is customized based on the status of a detected nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

The disclosed methods can include evaluating (such as quantifying) and/or interpreting at least one cell material released from a potential metastasis site (such as at least one cell material in a sample from a subject) and/or cell types that contribute to DNA, such as cfDNA, in one or more samples collected from a subject at one or more timepoints in comparison to a selected baseline value or reference standard (or a selected set of baseline values or reference standards). A baseline value or reference standard may be a presence or level of at least one cell material and/or a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a presence or level of at least one cell material and/or a quantity of cell types measured with respect to one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

The disclosed methods can include evaluating (such as quantifying) and/or interpreting one or more genetic and/or epigenetic signatures, and/or one or more cell types (such as one or more immune cell types), present in one or more samples (e.g., in DNA, such as cfDNA, from a blood sample (e.g., a whole blood sample, a buffy coat sample, a leukapheresis sample, or a PBMC sample)) collected from a subject at one or more timepoints in comparison to a selected baseline value or reference standard (or a selected set of baseline values or reference standards). A baseline value or reference standard may be a quantity of copy number variation, rare mutations, cancer-related epigenetic signatures (such as hypermethylated regions or hypomethylated regions), and/or cell types measured in one or more samples (such as an average quantity or range of quantities of such signatures present in at least two samples) collected from the subject at one or more time points, such as prior to receiving a treatment, prior to diagnosis of a condition (such as a cancer), or as part of a preventative health monitoring program. A baseline value or reference standard may be a quantity of, e.g., copy number variation, rare mutations, cancer-related epigenetic signatures (such as hypermethylated regions or hypomethylated regions), and/or cell types measured in one or more samples (such as an average quantity or range of quantities of such signatures and/or cell types present in at least two samples) collected at one or more timepoints from one or more subjects that do not have the condition (such as a healthy subject that does not have a cancer), one or more subjects that responded favorably to the treatment, or one or more subjects that have not received the treatment.

In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the genetic and/or epigenetic signature quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a buffy coat sample or any other sample comprising cells, such as a blood sample (e.g., a whole blood sample, a leukapheresis sample, or a PBMC sample) may be collected from a subject at two or more timepoints, to assess changes in cell types (such as changes in quantities of cell types) between the two timepoints. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

In some embodiments, methods are provided for monitoring a response (such as a change in disease state, such as a presence or absence of a metastasis in a subject, such as measured by assessing a presence or level of at least one cell material released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment or the course or stage of a condition (such as a cancer) in the subject is being monitored over time, genetic and/or epigenetic signatures, and/or cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples (such as one or more whole blood, buffy coat, leukapheresis, or PBMC samples) are collected from a subject at least once per week, such as on 1-4 days, 1-2 days, or on 1, 2, 3, 4, 5, 6, or 7 days per week. In certain embodiments, one or more samples are collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### Table of Sequences

The following table shows exemplary sequences provided herein.

| SEQ ID NO | |
|---|---|
| and | |
| description | Sequence |
| SEQ ID NO: 1 | MDYKDDDDKHMGGSDFPSCRCVEQIIEKDEGPFYTHLGAGPNVAAIREIMEERFGQKGKAI |
| wild type | RIERVIYTGKEGKSSQGCPIAKWVVRRSSSEEKLLCLVRERAGHTCEAAVIVILILVWEGI |
| TET2 | PLSLADKLYSELTETLRKYGTLTNRRCALNEERTCACQGLDPETCGASFSFGCSWSMYYNG |
| catalytic | CKFARSKIPRKFKLLGDDPKEEEKLESHLQNLSTLMAPTYKKLAPDAYNNQIEYEHRAPEC |
| domain | RLGLKEGRPFSGVTACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLVFYQHKSMNEPKHGLALWEAKMAEKAREKEEECEKYG |
| SEQ ID NO: 2 | MDYKDDDDKHMGGSDFPSCRCVEQIIEKDEGPFYTHLGAGPNVAAIREIMEERFGQKGKAI |
| TET2 V1900A | RIERVIYTGKEGKSSQGCPIAKWVVRRSSSEEKLLCLVRERAGHTCEAAVIVILILVWEGI |
| catalytic | PLSLADKLYSELTETLRKYGTLTNRRCALNEERTCACQGLDPETCGASFSFGCSWSMYYNG |
| domain | CKFARSKIPRKFKLLGDDPKEEEKLESHLQNLSTLMAPTYKKLAPDAYNNQIEYEHRAPEC |
| | RLGLKEGRPFSGVTACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLAFYQHKSMNEPKHGLALWEAKMAEKAREKEEECEKYG |
| SEQ ID NO: 3 | MDYKDDDDKHMGGSDFPSCRCVEQIIEKDEGPFYTHLGAGPNVAAIREIMEERFGQKGKAI |
| TET2 V1900C | RIERVIYTGKEGKSSQGCPIAKWVVRRSSSEEKLLCLVRERAGHTCEAAVIVILILVWEGI |
| catalytic | PLSLADKLYSELTETLRKYGTLTNRRCALNEERTCACQGLDPETCGASFSFGCSWSMYYNG |
| domain | CKFARSKIPRKFKLLGDDPKEEEKLESHLQNLSTLMAPTYKKLAPDAYNNQIEYEHRAPEC |
| | RLGLKEGRPFSGVTACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLCFYQHKSMNEPKHGLALWEAKMAEKAREKEEECEKYG |
| SEQ ID NO: 4 | MDYKDDDDKHMGGSDFPSCRCVEQIIEKDEGPFYTHLGAGPNVAAIREIMEERFGQKGKAI |
| TET2 V1900G | RIERVIYTGKEGKSSQGCPIAKWVVRRSSSEEKLLCLVRERAGHTCEAAVIVILILVWEGI |
| catalytic | PLSLADKLYSELTETLRKYGTLTNRRCALNEERTCACQGLDPETCGASFSFGCSWSMYYNG |
| domain | CKFARSKIPRKFKLLGDDPKEEEKLESHLQNLSTLMAPTYKKLAPDAYNNQIEYEHRAPEC |
| | RLGLKEGRPFSGVTACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLGFYQHKSMNEPKHGLALWEAKMAEKAREKEEECEKYG |
| SEQ ID NO: 5 | MDYKDDDDKHMGGSDFPSCRCVEQIIEKDEGPFYTHLGAGPNVAAIREIMEERFGQKGKAI |
| TET2 V1900I | RIERVIYTGKEGKSSQGCPIAKWVVRRSSSEEKLLCLVRERAGHTCEAAVIVILILVWEGI |
| catalytic | PLSLADKLYSELTETLRKYGTLTNRRCALNEERTCACQGLDPETCGASFSFGCSWSMYYNG |
| domain | CKFARSKIPRKFKLLGDDPKEEEKLESHLQNLSTLMAPTYKKLAPDAYNNQIEYEHRAPEC |
| | RLGLKEGRPFSGVTACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGSGGGGSDEVWSDSEQSFLDPDIGGVAVAPTHGSILIECAKRELHATTPLKNP |
| | NRNHPTRISLIFYQHKSMNEPKHGLALWEAKMAEKAREKEEECEKYG |
| SEQ ID NO: 6 | MDYKDDDDKHMGGSDFPSCRCVEQIIEKDEGPFYTHLGAGPNVAAIREIMEERFGQKGKAI |
| TET2 V1900P | RIERVIYTGKEGKSSQGCPIAKWVVRRSSSEEKLLCLVRERAGHTCEAAVIVILILVWEGI |
| catalytic | PLSLADKLYSELTETLRKYGTLTNRRCALNEERTCACQGLDPETCGASFSFGCSWSMYYNG |
| domain | CKFARSKIPRKFKLLGDDPKEEEKLESHLQNLSTLMAPTYKKLAPDAYNNQIEYEHRAPEC |
| | RLGLKEGRPFSGVTACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGSGGGGGGGGSDEVWSDSEQSFLDPDIGGVAVAPTHGSILIECAKRELHATTPLKNP |
| | NRNHPTRISLPFYQHKSMNEPKHGLALWEAKMAEKAREKEEECEKYG |
| SEQ ID NO: 7 | MNSNKDKIKVIKVFEAFAGIGSQFKALKNIARSKNWEIQHSGMVEWFVDAIVSYVA |
| Mycoplasma | IHSKNFNPKIERLDRDILSISNDSKMPISEYGIKKINNTIKASYLNYAKKHENNLF |
| penetrans | DIKKVNKDNFPKNIDIFTYSFPCQDLSVQGLQKGIDKELNTRSGLLWEIERILEEI |
| CpG | KNSFSKEEMPKYLLMENVKNLLSHKNKKNYNTWLKQLEKFGYKSKTYLLNSKNEDN |
| carboxymethyl- | CQNRERVFCLSIRDDYLEKTGFKFKELEKVKNPPKKIKDILVDSSNYKYLNLNKYE |
| transferase | TTTFRETKSNIISRPLKNYTTENSENYVYNINGIGPTLTASGANSRIKIETQQGVR |
| N374K | YLTPLECFKYMQFDVNDFKKVQSTNLISENKMIYIAGKSIPVKILEAIENTLEFVN |
| | NEELE |
| SEQ ID NO: 8 | MNSNKDKIKVIKVFEAFAGIGSQFKALKNIARSKNWEIQHSGMVEWFVDAIVSYVA |
| Mycoplasma | IHSKNFNPKIERLDRDILSISNDSKMPISEYGIKKINNTIKASYLNYAKKHENNLF |
| penetrans | DIKKVNKDNFPKNIDIFTYSFPCQDLSVQGLQKGIDKELNTRSGLLWEIERILEEI |
| CpG | KNSFSKEEMPKYLLMENVKNLLSHKNKKNYNTWLKQLEKFGYKSKTYLLNSKNEDN |
| carboxymethyl- | CQNRERVFCLSIRDDYLEKTGFKFKELEKVKNPPKKIKDILVDSSNYKYLNLNKYE |
| transferase | TTTFRETKSNIISRPLKNYTTENSENYVYNINGIGPTLTASGANSRIKIETQQGVR |
| N374R | YLTPLECFKYMQFDVNDFKKVQSTNLISENKMIYIAGRSIPVKILEAIENTLEFVN |
| | NEELE |
| SEQ ID NO: 9 | QSQNGKCEGCNPDKDEAPYYTHLGAGPDVAAIRTLMEERYGEKGKAIRIEKVIYTG |
| TETcd | KEGKSSQGCPIAKWVYRRSSEEEKLLCLVRVRPNHTCETAVMVIAIMLWDGIPKLL |
| | ASELYSELTDILGKCGICTNRRCSQNETRNCCCQGENPETCGASESFGCSWSMYYN |
| | GCKFARSKKPRKFRLHGAEPKEEERLGSHLQNLATVIAPIYKKLAPDAYNNQVEFE |
| | HQAPDCCLGLKEGRPFSGVTACLDFSAHSHRDQQNMPNGSTVVVTLNREDNREVGA |
| | KPEDEQFHVLPMYIIAPEDEFGSTEGQEKKIRMGSIEVLQSFRRRRVIRIGELPKS |
| | CKKKAEPKKAKTKKAARKRSSLENCSSRTEKGKSSSHTKLMENASHMKQMTAQPQL |
| | SGPVIRQPPTLQRHLQQGQRPQQPQPPQPQPQTTPQPQPQPQHIMPGNSQSVGSHC |
| | SGSTSVYTRQPTPHSPYPSSAHTSDIYGDTNHVNFYPTSSHASGSYLNPSNYMNPY |
| | LGLLNQNNQYAPFPYNGSVPVDNGSPFLGSYSPQAQSRDLHRYPNQDHLTNQNLPP |
| | IHTLHQQTFGDSPSKYLSYGNQNMQRDAFTTNSTLKPNVHHLATFSPYPTPKMDSH |
| | FMGAASRSPYSHPHTDYKTSEHHLPSHTIYSYTAAASGSSSSHAFHNKENDNIANG |
| | LSRVLPGENHDRTASAQELLYSLTGSSQEKQPEVSGQDAAAVQEIEYWSDSEHNFQ |
| | DPCIGGVAIAPTHGSILIECAKCEVHATTKVNDPDRNHPTRISLVLYRHKNLFLPK |
| | HCLALWEAKMAEKARKEEECGKNGSDHVSQKNHGKQEKREPTGPQEPSYLRFIQSL |
| | AENTGSVTTDSTVTTSPYAFTQVTGPYNTFV |
| SEQ ID NO: | GGSQSQNGKCEGCNPDKDEAPYYTHLGAGPDVAAIRTLMEERYGEKGKAIRIEKVIYTGKE |
| 10 TETV | GKSSQGCPIAKWVYRRSSEEEKLLCLVRVRPNHTCETAVMVIAIMLWDGIPKLLASELYSE |
| | LTDILGKCGICTNRRCSQNETRNCCCQGENPETCGASFSFGCSWSMYYNGCKFARSKKPRK |
| | FRLHGAEPKEEERLGSHLQNLATVIAPIYKKLAPDAYNNQVEFEHQAPDCCLGLKEGRPES |
| | GVTACLDFSAHSHRDQQNMPNGSTVVVTLNREDNREVGAKPEDEQFHVLPMYIIAPEDEFG |
| | STEGQEKKIRMGSIEVLQSFRRRRVIRIGELPKSCEVSGQDAAAVQEIEYWSDSEHNFQDP |
| | CIGGVAIAPTHGSILIECAKCEVHATTKVNDPDRNHPTRISLVLYRHKNLFLPKHCLALWE |
| | AKMAEKARKEEECGKNGSDHVSQKNHGKQEKREPTGPQEPSYLRFIQSLAENTGSVTTDST |
| | VTTSPYAFTQVTGPYNTFV |
| SEQ ID NO: | MDYKDDDDKHMGGSDFPSCRCVEQIIEKDEGPFYTHLGAGPNVAAIREIMEERFGQKGKAI |
| 11 | RIERVIYTGKEGKSSQGCPIAKWVVRRSSSEEKLLCLVRERAGHTCEAAVIVILILVWEGI |
| TET2-CS- | PLSLADKLYSELTETLRKYGTLTNRRCALNEERTCACQGLDPETCGASFSFGCSWSMYYNG |
| T1372S | CKFARSKIPRKFKLLGDDPKEEEKLESHLQNLSTLMAPTYKKLAPDAYNNQIEYEHRAPEC |
| | RLGLKEGRPFSGVSACLDFCAHAHRDLHNMQNGSTLVCTLTREDNREFGGKPEDEQLHVLP |
| | LYKVSDVDEFGSVEAQEEKKRSGAIQVLSSFRRKVRMLAEPVKTCRQRKLEAKKAAAEKLS |
| | GGGGGGGGSGGGGSDEVWSDSEQSFLDPDIGGVAVAPTHGSILIECAKRELHATTPLKNP |
| | NRNHPTRISLVFYQHKSMNEPKHGLALWEAKMAEKAREKEEECEKYG |
| SEQ ID NO: | MGGSDFPSCRCVEQIIEKDEGPFYTHLGAGPNVAAIREIMEERFGQKGKAIRIERVIYTGK |
| 12 | EGKSSQGCPIAKWVVRRSSSEEKLLCLVRERAGHTCEAAVIVILILVWEGIPLSLADKLYS |
| TET2-CD- | ELTETLRKYGTLTNRRCALNEERTCACQGLDPETCGASFSFGCSWSMYYNGCKFARSKIPR |
| T1372S | KFKLLGDDPKEEEKLESHLQNLSTLMAPTYKKLAPDAYNNQIEYEHRAPECRLGLKEGRPF |
| | SGVSACLDFCAHAHRDLHNMQNGSTLVCTLTREDNREFGGKPEDEQLHVLPLYKVSDVDEF |
| | GSVEAQEEKKRSGAIQVLSSFRRKVRMLAEPVKTCRQRKLEAKKAAAEKLSSLENSSNKNE |
| | KEKSAPSRTKQTENASQAKQLAELLRLSGPVMQQSQQPQPLQKQPPQPQQQQRPQQQQPHH |
| | PQTESVNSYSASGSTNPYMRRPNPVSPYPNSSHTSDIYGSTSPMNFYSTSSQAAGSYLNSS |
| | NPMNPYPGLLNQNTQYPSYQCNGNLSVDNCSPYLGSYSPQSQPMDLYRYPSQDPLSKLSLP |
| | PIHTLYQPRFGNSQSFTSKYLGYGNQNMQGDGFSSCTIRPNVHHVGKLPPYPTHEMDGHFM |
| | GATSRLPPNLSNPNMDYKNGEHHSPSHIIHNYSAAPGMENSSLHALHLQNKENDMLSHTAN |
| | GLSKMLPALNHDRTACVQGGLHKLSDANGQEKQPLALVQGVASGAEDNDEVWSDSEQSFLD |
| | PDIGGVAVAPTHGSILIECAKRELHATTPLKNPNRNHPTRISLVFYQHKSMNEPKHGLALW |
| | EAKMAEKAREKEEECEKYGPDYVPQKSHGKKVKREPAEPHETSEPTYLRFIKSLAERTMSV |
| | TTDSTVTTSPYAFTRVTGPYNRYI |
| SEQ ID NO: | FSGVTACLD |
| 13 | |
| SEQ ID NO: | FSGVSACLD |
| 14 | |
| TET2 T1372S | |
| SEQ ID NO: | MNPQIRNPMERMYRDTFYDNFENEPILYGRSYTWLCYEVKIKRGRSNLLWDTGVFRGQVYF |
| 15 | EPQYHAEMCFLSWFCGNQLPAYKCFQITWFVSWTPCPDCVAKLAEFLSEHPNVTLTISAAR |
| HYPER-A3B-1 | LYYYWERDYRRALCRLSQAGARVKIMDYEEFAYCWENFVYNEGQQFMPWYKFDENYAFLHR |
| | TLKEILRYLMEASPASGPRHLMDPHIFTSNENNGIGRHKTYLCYEVERLDNGTSVKMDQHR |
| | GFLHNQAKNLLCGFYGRHAELRFLDLVPSLQLDPAQIYRVTWFISWSPCFSWGCAGEVRAF |
| | LQENTHVRLRIFAARIYDYDPLYKEALQMLRDAGAQVSIMTYDEFKHCWDTFVDHQGCPFQ |
| | PWDGLDEHSQALSGRLRAILQNQGN |

### Kits

Also provided are kits comprising reagents for performing the methods as described herein. The kits can be for use in performing the methods as described herein. In some embodiments, a kit comprises a plurality of adapters.

In some embodiments, the kit comprises one or more conversion reagents. The conversion reagents may comprise reagents for any combination of steps described herein, including but not limited to in the numbered embodiments above and in any one of the workflows shown in the figures.

In some embodiments, a kit comprises a plurality of primers. In some embodiments, a kit comprises a plurality of capture probes. In some embodiments, the kit comprises a DNA polymerase. In some embodiments, the kit comprises deoxynucleoside triphosphates. In some embodiments, the kit comprises PCR primers that anneal to an adapter included in the kit. In some embodiments, the kit comprises additional elements described elsewhere herein. In some embodiments, the kit comprises instructions for performing a method described herein.

In some embodiments, a kit further comprises an agent that recognizes methyl cytosine in DNA. In some such embodiments, the agent is an antibody or a methyl binding protein or methyl binding domain. In some embodiments, the kit comprises capture probes that specifically bind to epigenetic and/or sequence-variable target region sets. In some such embodiments, the capture probes comprise a capture moiety. In some embodiments, the kit comprises a solid support linked to a binding partner of the capture moiety.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABLI, AKTI, ATM, CDHI, CSFIR, CTNNBI, ERBB4, EZH2, FGFRI, FGFR2, FGFR3, FLT3, GNA11, GNAQ, GNAS, HNFIA, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRKI. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The kit can comprise at least 4, 5, 6, 7, or 8 different library adapters having distinct molecular barcodes and identical sample barcodes. The sample barcode may include the modified nucleotide, such as 5mC. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the library adapters can be found throughout and are applicable to the kit. In some embodiments, the adapters comprise quality control nucleosides.

### Computer Systems

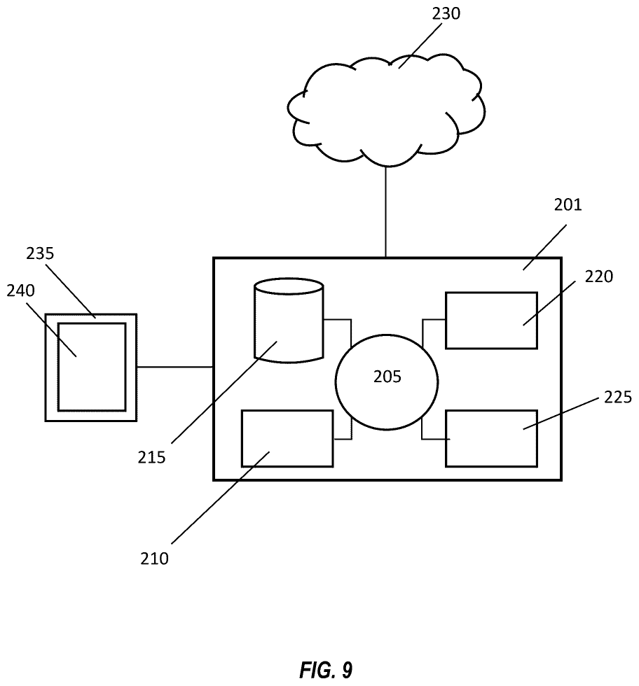

Methods of the present disclosure can be implemented using, or with the aid of, computer systems. FIG. 9 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including (where applicable) nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 201, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method described herein. For example, the method may comprise the steps of any one of the embodiments set forth in the introduction and summary section above.

The code can be pre-compiled and configured for use with a machine have a processor adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

Exemplary workflows for analyzing the modified nucleoside profile of nucleic acid in a sample and library preparation are provided herein. In some embodiments, some or all features of the partitioning and library preparation workflows may be used in combination.

Exemplary workflows are illustrated in FIGS. 1-8.

The workflows of FIG. 1 comprise contacting DNA with TET followed sequentially by contacting the DNA with β-glucosyltransferase (BGT) and/or contacting the DNA with TET a second time, followed by deamination (e.g., using an APOBEC such as APOBEC3A). In some embodiments, altering a buffer pH between the TET and BGT reactions, e.g., using a pH of 6-7 such as 6.5-6.8 for the TET reaction and a pH higher than 6.8 (e.g., 7.5-8.5, such as about 8), may improve sensitivity. This in turn is followed by library preparation PCR. The resulting library may then be sequenced.

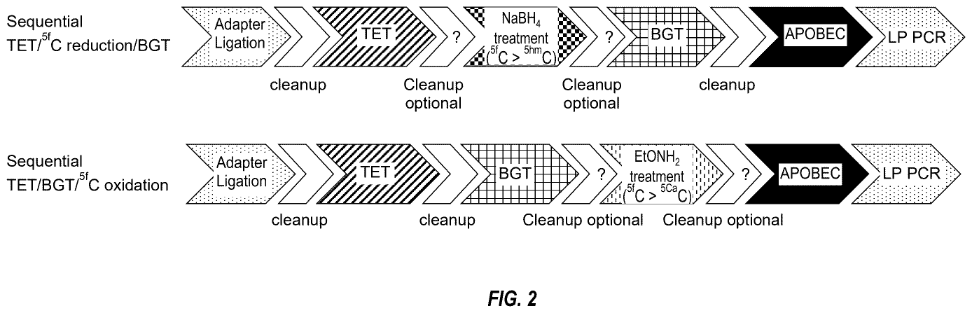

The workflows of FIG. 2 comprise contacting DNA with TET followed sequentially by contacting the DNA with NaBH₄, β-glucosyltransferase (βGT), and/or EtONH₂, followed by deamination (e.g., using an APOBEC such as APOBEC3A). This in turn is followed by library preparation PCR. The resulting library may then be sequenced.

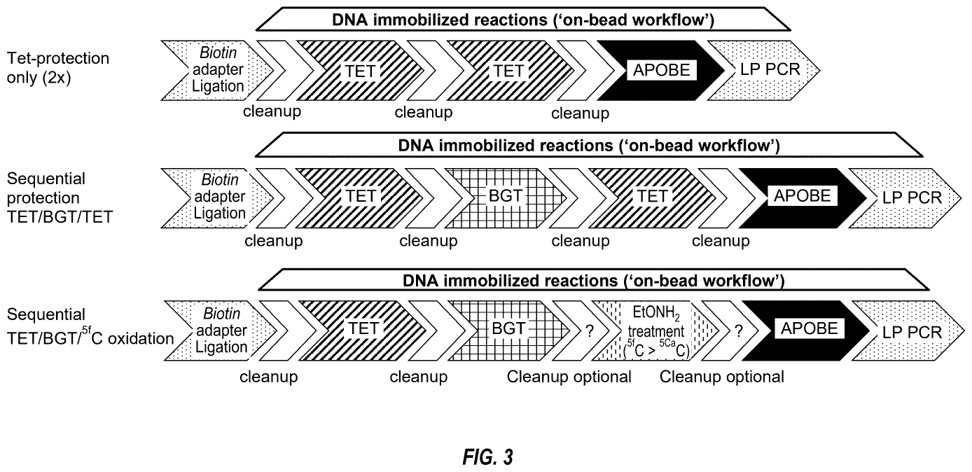

The workflows of FIG. 3 comprise contacting immobilized DNA with reagents such as TET and one or more of contacting the DNA with BGT, an additional step of contacting the DNA with TET, and contacting the DNA with EtONH₂, followed by deamination (e.g., using an APOBEC such as APOBEC3A). This in turn is followed by library preparation PCR. The resulting library may then be sequenced. Library immobilization can enable buffer exchange between enzyme reactions, thereby improving efficiencies.

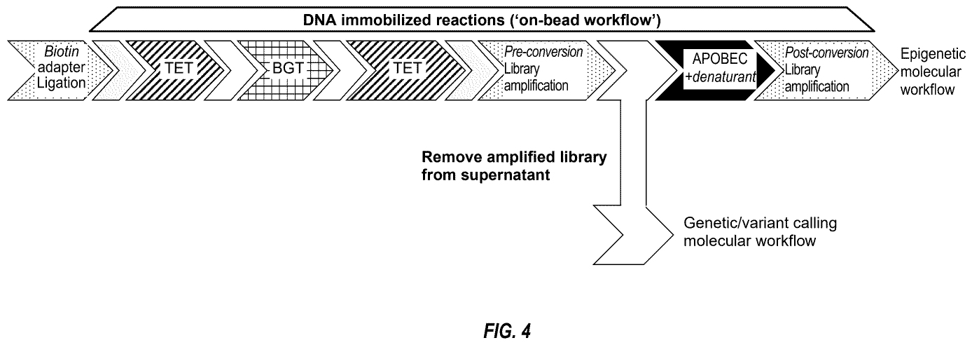

The workflow of FIG. 4 comprises contacting immobilized DNA with TET, then BGT, then TET again, wherein an amplified library is produced prior to treatment of the immobilized DNA with a deaminase (e.g., an APOBEC such as APOBEC3A). The pre-conversion amplified library is intended for calling of genetic variants such as point mutations, indels, etc. The immobilized DNA is subjected to deamination (e.g., using an APOBEC such as APOBEC3A). This in turn is followed by library preparation PCR. The resulting pre-conversion and post-conversion libraries may then be sequenced.

The workflow of FIG. 5 comprises contacting DNA with TET followed sequentially by contacting the DNA with β-glucosyltransferase (BGT) such as T4 BGT and then with EtONH₂, followed by deamination (e.g., using an APOBEC such as APOBEC3A). This in turn is followed by amplification (e.g., library preparation PCR) and sequencing.

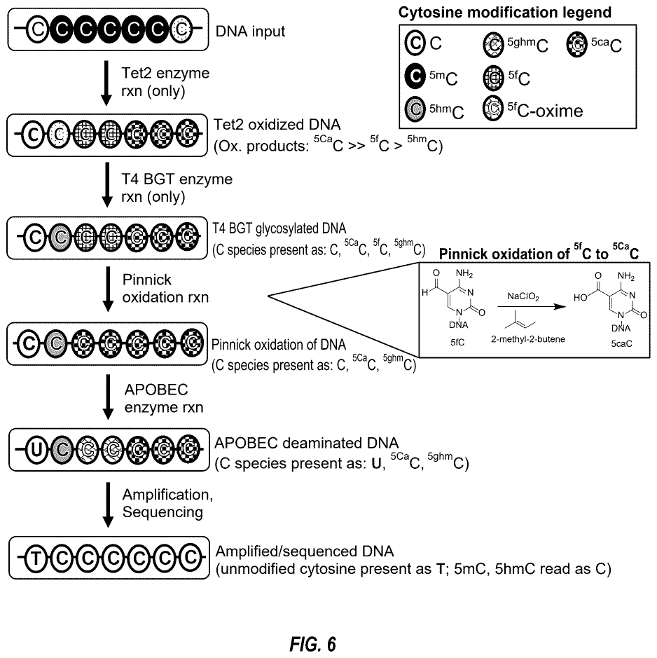

The workflow of FIG. 6 comprises contacting DNA with TET followed sequentially by contacting the DNA with β-glucosyltransferase (BGT) such as T4 BGT and then with Pinnick oxidation reagents, followed by deamination (e.g., using an APOBEC such as APOBEC3A). This in turn is followed by amplification (e.g., library preparation PCR) and sequencing.

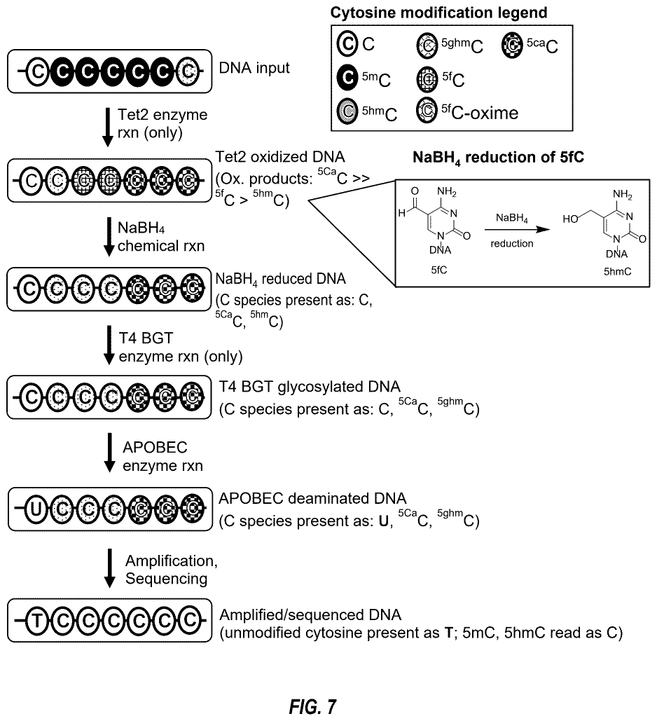

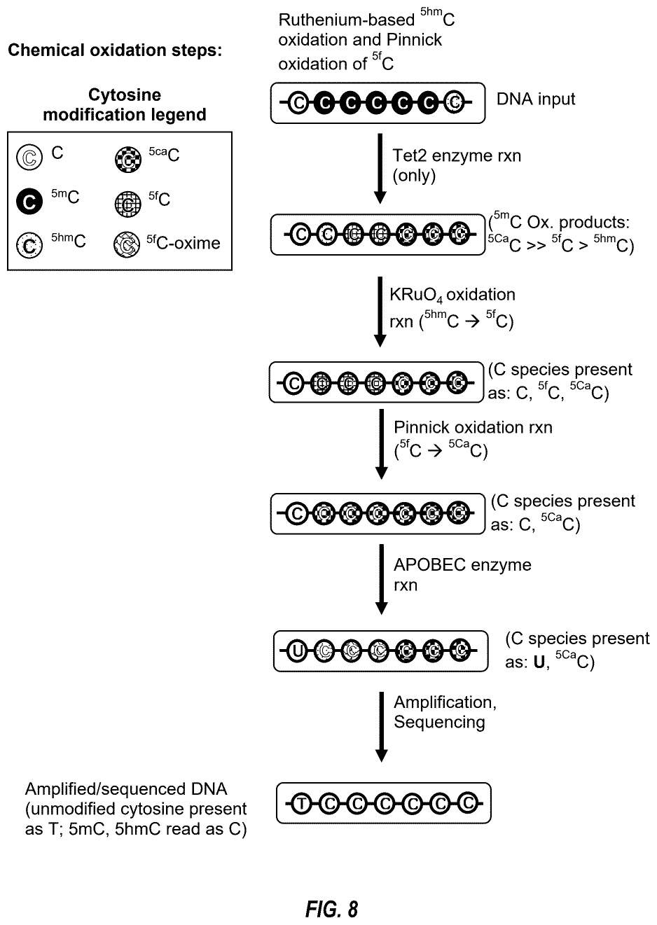

The workflow of FIG. 7 comprises contacting DNA with TET followed sequentially by contacting the DNA with NaBH₄ and then with β-glucosyltransferase (BGT) such as T4 BGT, followed by deamination (e.g., using an APOBEC such as APOBEC3A). This in turn is followed by amplification (e.g., library preparation PCR) and sequencing.

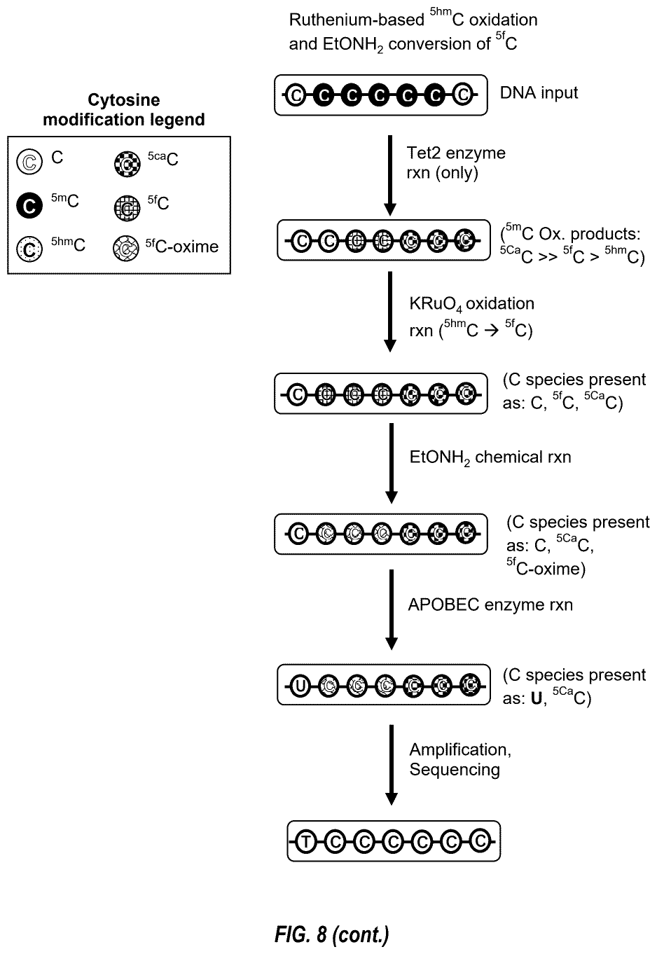

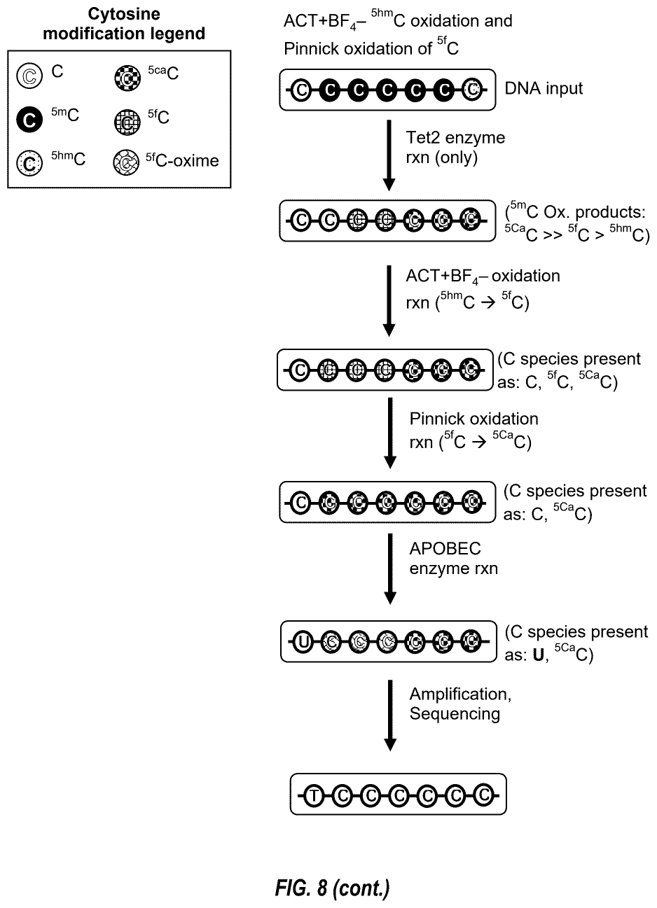

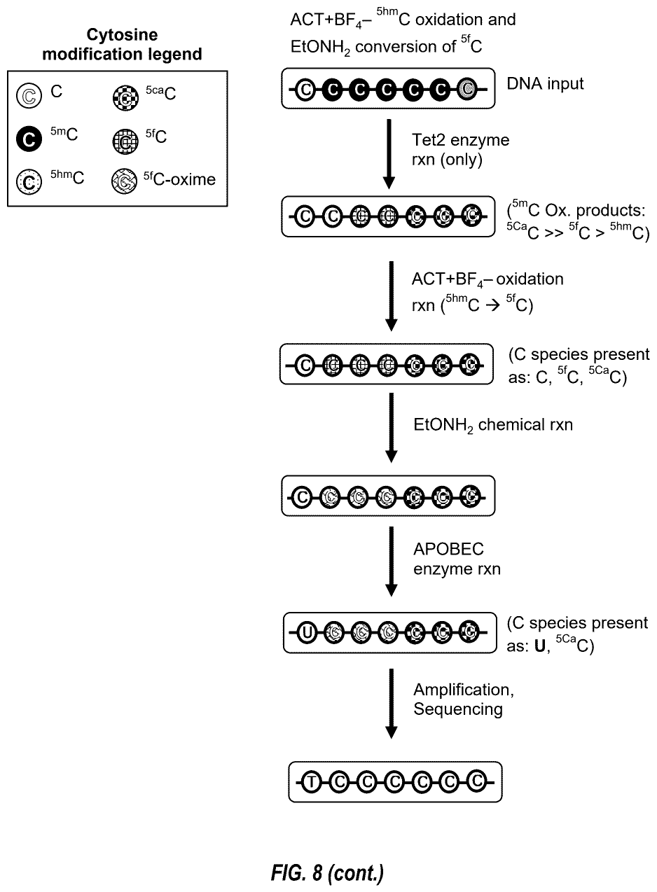

The workflow of FIG. 8 comprises contacting DNA with reagents such as TET followed sequentially by reagents selected from KRuO₄, Pinnick oxidation reagents, EtONH₂, and ACT⁺BF₄⁻. This in turn is followed by amplification (e.g., library preparation PCR) and sequencing.

1. A method comprising:
a) contacting DNA with one or more TET enzymes to oxidize 5-methylcytosine (5mC) present in the DNA, wherein, for at least a portion of the 5mC, the oxidation of 5mC results in formation of 5-formylcytosine (5-fC) for at least a portion of the 5mC;
b) after a), subjecting the DNA to a further treatment that reduces the amount of 5fC in the DNA and/or protects 5-hydroxymethylcytosine that may be present in the DNA;
c) contacting at least a portion of the DNA with a cytosine deaminase, thereby converting unmethylated cytosine in the DNA to uracil, thereby producing treated DNA; and
d) sequencing at least a portion of the treated DNA.

2. The method of claim 1, wherein the further treatment comprises:
(i) contacting the DNA with β-glucosyltransferase (βGT);
(ii) contacting the DNA with β-glucosyltransferase (βGT) and then contacting the DNA with one or more TET enzymes to further oxidize the 5fC;
(iii) contacting the DNA with βGT and then contacting the DNA with an alkoxylamine, such as a C₂₋₆ alkoxylamine, such as ethoxylamine (EtONH₂);
(iv) contacting the DNA with a ruthenate such as KRuO₄ and then contacting the DNA with an alkoxylamine, such as a C₂₋₆ alkoxylamine, such as EtONH₂;
(v) contacting the DNA with a reducing agent such as NaBH₄ and then contacting the DNA with βGT;
(vi) contacting the DNA with βGT and then contacting the DNA with Pinnick oxidation reagents;
(vii) contacting the DNA with a ruthenate such as KRuO₄ and then contacting the DNA with Pinnick oxidation reagents;
(viii) contacting the DNA with 4-acetamido-2,2,6,6-tetramethylpiperidine-1-oxoammonium tetrafluoroborate (ACT⁺BF₄⁻) and then contacting the DNA with Pinnick oxidation reagents;
(ix) contacting the DNA with 4-acetamido-2,2,6,6-tetramethylpiperidine-1-oxoammonium tetrafluoroborate (ACT⁺BF₄⁻) and then contacting the DNA with Pinnick oxidation reagents, optionally wherein the Pinnick oxidation reagents comprise a chlorite salt and an alkene, further optionally wherein the chlorite salt is sodium chlorite and/or the alkene is a branched C₅₋₁₀ alkene, such as a branched C₅₋₇ alkene, such as 2-methyl-2-butene;
(x) contacting the DNA with 4-acetamido-2,2,6,6-tetramethylpiperidine-1-oxoammonium tetrafluoroborate (ACT⁺BF₄⁻) and then contacting the DNA with an alkoxylamine, such as a C₂₋₆ alkoxylamine, such as EtONH₂;
(xi) contacting the DNA with one or more TET enzymes a second time after step a), wherein the method further comprises purifying the DNA between steps a) and b), optionally wherein the DNA is purified using solid phase reversible immobilization (SPRI); or
(xii) contacting the DNA with one or more TET enzymes a second time after step a) and then contacting the DNA with β-glucosyltransferase (βGT).

3.-13. (canceled)

14. The method of claim 1, wherein the one or more TET enzymes comprise TETv, TETcd, TET1, and/or TET2, optionally wherein the one or more TET enzymes comprise TET1 and TET2.

15.-18. (canceled)

19. The method of claim 1, wherein the DNA is contacted with the one or more TET enzymes in the absence of βGT, optionally wherein the DNA is contacted with βGT after it was contacted with the one or more TET enzymes.

20.-22. (canceled)

23. The method of claim 1, wherein the DNA is contacted with the one or more TET enzymes in a buffer having a pH ranging from 6 to 7.

24.-25. (canceled)

26. The method of claim 1, wherein the cytosine deaminase is an AID/APOBEC family cytosine deaminase enzyme, such as an APOBEC enzyme, optionally wherein the APOBEC enzyme is APOBEC3A or HYPER-A3B-1.

27. (canceled)

28. The method of claim 1, wherein the cytosine deaminase is MsddA or AshDa01.

29. (canceled)

30. The method of claim 1, wherein the DNA comprises a first member of a binding pair, optionally wherein the first member of the binding pair is biotin, further optionally wherein the first member of the binding pair is attached to an adapter joined to the DNA.

31. (canceled)

32. The method of claim 30, wherein the DNA is bound to a solid phase comprising a second member of the binding pair during steps a)-b), optionally wherein the solid phase comprises beads and/or the second member of the binding pair is streptavidin, further optionally wherein the DNA is released from the solid phase before step c).

33. (canceled)

34. The method of claim 32, wherein the method further comprises amplifying the DNA between step (b) and step (c), and prior to a step of releasing the DNA from the solid phase, optionally wherein the method further comprises separating the amplified DNA from the DNA bound to the solid phase prior to step (c), thereby contacting the DNA bound to the solid phase, but not the amplified DNA, with a cytosine deaminase in step (c), further optionally wherein the DNA bound to the solid phase is released from the solid phase prior to contacting the DNA with the cytosine deaminase.

35.-36. (canceled)

37. The method of claim 34, further comprising comparing sequence data obtained in step (d) with sequence data obtained by sequencing the amplified DNA that was not contacted with the cytosine deaminase; and identifying point differences between the converted DNA sequences and the or non-converted DNA sequence data as nucleosides having a modification status that permits a change in base pairing specificity on exposure to the cytosine deaminase, optionally wherein the DNA is released from the solid phase by cleaving a cleavable moiety in an adapter joined to the DNA, optionally wherein the cleavable moiety comprises uracil or photocleavable biotin.

38. (canceled)

39. The method of claim 1, wherein the method further comprises enriching the DNA by capturing a target region set from the sample, wherein the capture step is before, after or in between step (a) and step (c).

40. The method of claim 1, further comprising
a) comparing the sequence data obtained in step (d) with
(A) a pre-determined reference sequence; and/or
(B) sequence data obtained by sequencing a sub-sample of the DNA that was not subjected to a conversion procedure; and
b) identifying point differences between the converted DNA sequences and the reference sequence (A) or non-converted DNA sequence data (B) as nucleosides having a modification status that permits a change in base pairing specificity on exposure to the cytosine deaminase.

41. The method of claim 1, wherein the DNA comprises cell-free DNA (cfDNA), optionally cfDNA obtained from a test subject, optionally wherein the test subject is a patient having or suspected of having cancer, further optionally wherein the sample is a blood sample.

42. The method of claim 1, further comprising using the detection of modified nucleosides in the DNA sample to determine or predict the presence of DNA produced by a cancer cell or tumor, to determine the probability that a test subject has a tumor or cancer, or to characterize a cancer or tumor of the subject.

43. The method of claim 1, wherein a non-converted subsample of the DNA is not contacted with the cytosine deaminase before sequencing, wherein the treated subsample and the non-converted subsample have different adapter sequences, and wherein the converted subsample and the non-converted subsample are recombined before sequencing step (d).

44. (canceled)

45. The method of claim 1, further comprising analyzing the DNA to detect methylation and/or one or more of copy number variation, single nucleotide variants, insertions, deletions, and/or fusions.

46. The method of claim 1, further comprising capturing epigenetic target regions from the adapter-ligated DNA and amplifying and sequencing the epigenetic target regions, wherein the captured epigenetic target regions form an epigenetic target region set, optionally wherein the epigenetic target region set comprises a plurality of type-specific epigenetic target regions, and wherein the type-specific epigenetic target regions are type-specific differentially methylated regions and/or type specific fragments, further optionally wherein the plurality of type-specific epigenetic target regions comprises type-specific hypomethylated regions or type-specific hypermethylated regions, and further optionally wherein the type-specific epigenetic target regions comprise cell-type specific, tissue-type specific, and/or cancer-type specific epigenetic target regions.

47.-56. (canceled)

57. The method of claim 41, wherein the cell-free DNA is partitioned into a plurality of partitioned subsamples prior to capturing at least an epigenetic target region set of DNA.

58. The method of claim 1, further comprising detecting the presence or absence of sequence variations and/or determining fragmentation patterns, wherein adapted DNA comprising quality control nucleosides indicative of sub-optimal or erroneous conversion of quality control nucleosides is included in detecting the presence or absence of sequence variations and/or determining fragmentation patterns.

59. The method of claim 1, wherein oligonucleotide adapters are ligated to the DNA, optionally wherein the oligonucleotide adapters are ligated to the DNA before step a), further optionally wherein the oligonucleotide adapters comprise sequencing primer binding sites.

60.-61. (canceled)

62. The method of claim 59, wherein the method further comprises amplifying the DNA using primers targeting the adapters, wherein the amplifying step is between step (c) and the sequencing step (d).

63. The method of claim 59, wherein one or more nucleosides of the adapter is a modified nucleoside, optionally wherein the modified nucleoside comprises a modified cytosine, further optionally wherein the modified nucleoside is 5-carboxyl cytosine, pyrrolo cytosine, or 5-propynyl cytosine.

64. (canceled)
